(19) United States
(12) Patent Application Publication
(10) Pub. No.: US 2002/0010390 A1
(43) Pub. Date: Jan. 24, 2002

(54) Method and system for monitoring the health and status of livestock and other animals

(76) Inventors: David Lehmann Guice, Brownsboro, AL (US); Warren Ames Pugh, Weatherford, TX (US); Noble A. Thompson, Huntsville, AL (US)

Correspondence Address: Mark Clodfelter, Suite 1602D, 555 Sparkman Drive, Huntsville, AL, 35816, US

(21) Appl. No.: 09/853,460

(22) Filed: May 10, 2001

Related U.S. Application Data
(60) Provisional application No. 60/203,321, filed on May 10, 2000.

(51) Int. Cl. A61B 5/00

(52) U.S. Cl. 600/300

(57) ABSTRACT

A method and system (i.e., an Automated Animal Health Monitoring System—AAHMS) for automated monitoring and early warning of changes in parameters related to the health and status of animals is disclosed. The system includes implantable wireless “smart tele-sensor” elements that can be implanted within the animal where they measure, and may transmit, temperature and other parameters (e.g., blood oxygen, accelerations, vibrations, heart rate) related to the health and status of the animal being monitored. Optional relay elements may comprise simple transponders to boost the signals from the smart sensor elements and retransmit processed results. The system includes devices for alerting personnel responsible for care of the animals and identifying the animal needing attention. Installation tools include optional capabilities to program the smart sensor elements to adapt to animal type, season, diet, or other user needs, and to read and correlate electronic and machine read data with human readable animal identification (e.g., ear or collar tags).

### CROSS-REFERENCE TO RELATED APPLICATION

This application claims the benefit of U.S. provisional patent application No. 60/203,321, filed May 10, 2000.

### FIELD OF THE INVENTION

This application pertains to monitoring health and status of cattle and other animals, and particularly to a system wherein sensors implanted in the animals selectively provide temperature, location, and other parameters related to the animals health.

### BACKGROUND OF THE INVENTION

Problem/Opportunity to be Addressed

The present invention, in different embodiments, will have application in many aspects of animal product production and animal care environments where monitoring for animal health and status is needed. For illustrative purposes, we discuss herein an application in the livestock production industry, specifically cattle feedlots, but this discussion in no way limits the applications of the present invention in other areas where monitoring for animal health and status is needed.

Each year, about 35 million cattle are slaughtered in U.S. Approximately 28 million go through cattle feedlot operations for finishing before slaughter. Additional millions of swine and goats are also produced and slaughtered each year. In addition to animal products resulting from the slaughter of animals, other products produced from living animals include milk, and products derived from milk and eggs and wool and similar products derived from animal hair or fur. In the medical field, animals are used in the production of antibodies, insulin, and other medical products. Animals are also used in the production of other animals for companion animals, sports, utility animals, or other animal production environments. Many animals have high value for other reasons (e.g., companion animals, utility animals, endangered species, race horses).

Considerable human labor is expended each year in monitoring the health and status of animals in production environments as well as animals in other animal care environments (e.g., veterinary offices, research laboratories, stables, kennels, private environments). Even with the expenditure of considerable human labor in monitoring the health and status of animals, opportunities for improving the effectiveness of animal health monitoring exist as numerous animals still die or suffer disease progressions from which they are unable to completely recover. Failure to identify and isolate sick animals can also reduce overall efficiency in the production environment resulting from spread of disease pathogens to other animals, such spreading may be preventable with earlier detection than is provided by normal human monitoring.

In effect, those individuals or organizations overseeing such animal product production environments as feedlots and breeder barns are basically overseeing an organic chemical production environment. Just as instrumentation to monitor production processes is employed within a chemical production plant, such as an oil refinery or a plastics manufacturing plant, instrumentation systems are needed within animal production environments to enable more efficient and effective monitoring and control of the animal product production process while minimizing the costs of labor and other costs of production. This instrumentation may include instrumenting the animals themselves to monitor conditions important to the production efficiency for animal products. Similar gains in efficiency and effectiveness are also needed in other animal care environments.

One of the key conditions which can disrupt efficiency of animal product production environments is disease caused by pathogens, or other illness conditions, such as scours in calves caused by dietary deficiencies or imbalance. Another condition important to those animal production environments which are producing other animals, such as breeder barns for sows, is the onset of estrus in the females. Other conditions important to production efficiency including food conversion efficiency include environmental stresses such as hyperthermia caused by exposure to high heat conditions and direct sunlight, or hypothermia caused by exposure to low temperatures and wind conditions. Environmental effects on production efficiency may be more severe if coupled with disease conditions which interfere with performance of the animal's temperature regulation mechanisms. Other conditions potentially important to animal production efficiency include entanglement, entrapment, ingestion of toxic plants or inedible objects, undue exposure to predatory insects or other predators, or the occurrence of an injury that reduces the animal's mobility or causes other stress.

Importance of Early Detection of Illness

Early detection, isolation, and treatment of sick animals is critical to the profitability of feedlot, dairy, and other animal product production operations, as well as to the quality of, and selling price of, carcasses. Feedlot operations and other animal production environments frequently involve concentration of stressed animals from different locations, increasing likelihood of rapid spread of contagious diseases if sick animals are not identified early and separated from healthy animals. Before arriving at the feedlot, many animals may have spent two or three days in transit crowded together with other animals on trucks or trains, or in sales barns with little or no food and water, further adding to their stress and exposure to pathogens.

In the cattle industry, steers 10 are typically approximately nine months to one year old when they are shipped to a feedlot operation, and they may remain in the feedlot environment anywhere from 90 days up to one year, with most being in the feedlot for approximately 120 to 150 days. Typical feedlot operations have rectangular pens 12 which may range from 100 to 300 feet, more or less, in length on the sides. Pens may be adjacent as illustrated in FIG. 1. Although not explicitly illustrated in FIG. 1, as many as 200 or more cattle may be included in each pen. A large feedlot operation may have as many as 50,000 to 100,000 head of cattle on the premises at any one time. Feedlot pens also have feed bunks, watering troughs, gates, and other fixtures not shown in FIG. 1.

For individual animals, periods of illness reduce food conversion efficiency and carcass quality. If illness caused by one pathogen is not detected early, the stress of that illness may weaken the animal's immune system, leaving the animal susceptible to other pathogens in the production environment which are normally resisted by a healthy animal. For example, a viral infection may increase an animal's susceptibility to infection by bacteria which are normally present in the production environment but which are resisted by a healthy animal. Thus, lack of timely detection and intervention for individual sick animals can lead to use of more antibiotics and other medications than might be required with earlier detection and intervention, especially since additional pathogens may take advantage of the weakened animal. Furthermore, toxins produced by uncontrolled bacterial infections, as well as side effects of antibiotics and other medications, can damage organs and other tissues and significantly delay or prevent an animal from returning to normal food conversion efficiency and weight gain. Delays in detection of sick animals also increase the risk of death, resulting in major economic loss.

As illustrated in FIG. 2, cattle in feedlot operations which remain healthy, or those which develop illness but recover and remain free of antibiotics and other medications for prescribed periods, are generally shipped when they reach their target weight range to packing houses where they are slaughtered and their carcasses processed into meat products for human consumption. Animals that develop illness but fail to completely recover, or fail to develop sufficient weight, are generally shipped to specialty markets or, in some cases, put back out to grass pasture. Animals that do not recover adequately, and some which die on the lot, are generally sold to other packers and processed for pet food. Others who die may simply be taken to landfills, and the feedlot operator may have to pay to have the animal hauled or accepted in the landfill.

In some cases, the lack of an economically effective, automated means for monitoring the health of animals has led feedlot operators and other animal producers to widespread use (via injections or mixing with feed) of antibiotics and other medications in an attempt to prevent infections and reduce illness within their animals. However, many of the same or similar pathogens infect humans, and there is increasing concern in the human medical community that such widespread use of antibiotics, which frequently are the same as used in treatment of human illnesses, is leading to resistance buildup in the targeted pathogens and in other pathogens. When these resistant pathogens are transmitted to humans via the animal products and by other means, the result can be a general loss of effectiveness of these antibiotics in the treatment of humans.

In many cases, the antibiotics themselves also cause damage to the animal's tissue at the injection site. Damaged areas from multiple injections must be cut out during carcass processing, causing loss of product, increased processing labor, and consequently reduced grade-out and selling price for the carcasses.

Environmental effects such as heat waves and blizzards can also lead to loss of animals or loss in production efficiency due to hyperthermia or hypothermia. Some animals are less capable of dealing with environmental stress than other animals in the same environment. An automated animal health monitoring system (AAHMS) should be effective in detecting hypothermia as well as hyperthermia.

Monitoring for Estrus

Separate and apart from animal feedlot operations, there is also a need in many different types of animals to monitor for estrus. In production animals such as beef and dairy cattle, swine, and goats, there are significant economic losses associated with missing a breeding or artificial insemination opportunity within the optimum period. Similar needs (to not miss a breeding or artificial insemination opportunity) exist with other animals including horses, rare or endangered species, and utility or companion animals (pets). In most mammals and in some other animals, the onset of estrus is accompanied by a detectable increase in body temperature or change in the pattern of daily temperature variations. The onset of estrus is also accompanied by behavioral changes and the pattern of movement in some animals.

Monitoring for Pregnancy (Successful Breeding)

In addition to monitoring for the onset of estrus, there is also a need to determine whether production animals are pregnant (i.e., when breeding attempts have been successful). In swine production, for example, the diet of breeder sows is modified depending upon whether the sow is pregnant or whether the sow must be “maintained” to await another breeding attempt. Early knowledge of breeding success is also important to resource planning and management.

The onset of pregnancy is likely to be accompanied by measurable changes in the diurnal temperature pattern of mammals, as well as blood flow and distribution of body temperature.

Current Practices

The current practice for detection of sick animals in most cattle feedlot operations is to employ “pen-rider” cowboys who ride about the stock pens looking for cattle that show evidence of being sick. The surveillance techniques rely on such traits as runny nose, head down, or general reduced mobility and alertness to identify animals that may need attention. Sick animals so identified are then generally separated from the other animals in the pen and taken to a hospital pen for treatment.

The cattle industry is having increased difficulty in finding “pen-rider” cowboys willing to put up with the working conditions for the pay that feedlot operators can pay and remain economically viable. Furthermore, even good pen-rider cowboys miss timely detection of some sick animals, with the result that sick cattle are not treated in a timely manner leading to productive recovery. Also, typical feedlot industry losses to death range from one (1) to three (3) percent of animals which enter feedlot operations.

There are some approaches for monitoring for estrus in cows that have achieved at least partial commercial success. One approach uses a patch that is glued onto the cow's back just forward of the cow's tail. The patch contains one or more breakable vials of highly visible dye. If the cow enters estrus and is mounted by a “jump” bull or another animal, the vials break, releasing dye on the rear of the cow. This visible dye may then be observed by a “pen-rider” cowboy or other personnel during their daily rounds. Another approach Heat Watch™ for estrus monitoring also uses a similarly located patch, but in this alternate approach, the patch contains a pressure sensitive switch and a radio transmitter. When the cow is mounted, a switch is closed and a signal is transmitted to receivers mounted in the vicinity of the stock pen. The receivers are connected to a computer that then displays a message to personnel responsible for care of the herd. This approach may provide more timely and comprehensive detection, but still requires that the cow be mounted to trigger the alarm. It is also difficult in some conditions to keep the patch glued onto the cow's rump.

Prior Art, Limitations, and Opportunities for Innovation

There have been a series of attempts over the past twenty years or more to develop an effective means for monitoring the temperature of animals. The need for such monitoring has been widely recognized. However, as of this writing, none of the approaches described in prior art have reached significant commercial application in the feedlot industry, or in other segments of the animal or animal product production industries. We believe that a key reason for lack of acceptance of prior art has been the lack of a complete system solution which addresses all the requirements and constraints on those in the animal and animal product production industries, and which is practical and economical for use in a large scale production environment, such as a feedlot.

For example, U.S. Pat. No. 3,781,837 describes a temperature-measuring device which is installed into a cow's ear and held on by straps. This device required temperature compensation in order to monitor animal temperature relative to ambient temperature, but provided no means for sending an alert to a central control point, which is important for large scale production operations.

U.S. Pat. No. 3,893,111 describes an apparatus and method for remotely monitoring an animal's temperature, but does not describe a practical approach for scaling the method to large-scale operations such as commercial feedlots.

U.S. Pat. No. 4,399,821 describes an animal physiological monitoring and identification system with options for monitoring several different physiological parameters. However, the alerting technique described in this patent, when abnormal conditions are detected, consists of stimulating the animal to provoke and observable response, such as muscle twitching. It would be impractical to rely on this alert response in a large-scale commercial operation.

U.S. Pat. No. 4,844,076 describes an ingestible continuously transmitting temperature monitoring pill. However, the range for this device is not practical for use in large-scale commercial operations, nor does it have any features for conserving battery life for long-term operation.

U.S. Pat. No. 4,854,328 describes an implantable temperature monitoring electronic capsule with a small low power transmitter. However, the range of this device is limited and requires a receiver attached to the animal. Although this patent discloses the use of a relay device for increased range, no means are provided to avoid collisions between simultaneous transmissions from multiple animals, and no features are described to achieve the long battery lifetime required for commercial feedlot operations.

U.S. Pat. No. 4,865,044 discloses a temperature sensing system for cattle that uses a transmitter and encoding circuitry mounted an ear tag connected by wire to a temperature-sensing probe located in the ear canal of the animal being monitored. Although this system has value in monitoring temperature of animals in research or small volume operations, there will likely be problems with installation time and with retention of the wired probe in the ear canal when this system is used in large-scale commercial applications.

U.S. Pat. Nos. 5,984,875, 6,059,733, and 6,099,482 describe an animal temperature sensor system that uses ingestible boluses for monitoring physiological parameters of animals. Although the size of these boluses provides for a longer battery life, and the boluses contain an RF transmission capability, they present a risk related to contamination of food products if they are not located and recovered during slaughter and processing of food animals.

U.S. Pat. No. 6,113,539 describes a physical monitoring system for feedlot animals. This system uses a removable monitoring sleeve attached around an appendage of the animal, with the preferred appendage being the tail. Included in the monitoring sleeve is an instrument pack, one or more biosensors capable of measuring various physiological parameters, and optionally an RF transmitter. When abnormal conditions are detected, an alert can be transmitted to a central monitor computer, and the feedlot operator can scout for and render assistance to the animal that triggered the alert. Scouting is assisted by use of a light on the instrument pack that can be activated by radio command from the feedlot operator. Optional embodiments described therein include use of a radiolocation capability, or a GPS receiver, to monitor animal movements as an indicator of sickness. This patent teaches against the use of implants due to alleged risk of infection. However, we believe that it well be difficult to install and maintain the removal sleeves taught by this patent in a commercial feedlot environment. Additionally, the temperatures and other physiological parameters monitored by sensors mounted external to the animal are not likely to be as reliable as parameters measured by sensors installed within the animal's tissue are within cavities in the animal, such as the ear canal. Furthermore, this patent does not disclose all elements of an integrated system, such as automated alerting of the pen rider, and correlation of electronic ID with human readable ID, and other elements and features needed for efficient commercial operations.

None of these systems in the current art provides for a monitoring system that is complete, practical, and cost effective for use in commercial feedlot operations.

Systems Approach

The lack of availability of an effective, automated animal health monitoring system (AAHMS) is due, in part, to the absence of an innovative “systems” approach to providing such a capability in the cattle feedlot industry and in other areas where animal health monitoring is needed.

It is one object of the present invention to provide cost-effective and efficient methods and systems for automated monitoring of the health and status of animals in both large-scale animal production operations as well as in other animal care environments where diligence in monitoring animal health is required. It is another object of the invention to provide methods and tools for installing the sensors and other system elements into monitored animals and into the animal production environment and other animal care environments. It is yet another object of the present invention to provide, in most applications and embodiments, substantially more effective monitoring of the health and status of animals than is typically provided by present methods employed in present animal product production environments and other animal care environments, and to provide such enhanced monitoring, in most applications and embodiments, with a reduction in overall labor typically required for effective operation of said animal product production or other animal care environments. It is a further object of the present invention, in some embodiments, to provide a capability for early detection of sick animals, and to provide a means for users of the present invention to adjust operational parameters or algorithms used by the AAHMS elements to control the tradeoff between maintaining adequate probability of early detection of sick animals and keeping false alarm rates low enough to avoid waste of labor in responding to false alarm conditions. It is an additional object of the present invention, in certain embodiments, to reduce the time needed to locate the specific animal from which an alarm condition originates in the presence of many other animals (e.g., in a feedlot, corral, or pasture) and provide for visual is identification by caretakers to insure the proper animal is selected for isolation, treatment, or closer monitoring. It is an additional object of the present invention to further support early intervention and reduce labor (1) by providing for earlier identification of abnormal conditions, in some applications and embodiments, than is provided by present methods and (2) by providing for automated, timely notification of an appropriate attendant or caregiver, using means appropriate to the animal production environment, such as automated transmission or relay of alert data to digital pagers, use of synthesized voice to provide alert information to appropriate personnel over personal hand-operated radios (e.g., walkie-talkies) or telephones, or transfer of data to Personal Digital Assistants (PDAs) carried by animal attendants. It is a further object of the present invention to provide for automated capture, analysis, and display of information which may be valuable in the diagnosis of sick or stressed animals to help identify cause of illness or stress and support selection of appropriate treatments and specific antibiotics or other medications to be administered. This includes, for example, a capability to store and provide an animal's temperature history at various times, for example, hourly intervals over a three day period, to aid in the diagnosis of the animal's condition and identification of causative agent. Optionally, the overall system may include innovative devices for identification of specific pathogens (e.g., employing various chemical, biological, or electronic means for identifying specific biological agents, antibodies, or DNA). It is a further object of the present invention to provide, in at least some embodiments, a capability to acquire and/or use information about the ambient environment parameters (e.g., temperature, humidity, rainfall, wind, dust) which have an effect upon the physiology or other conditions of the animal(s) being monitored. Given that meat for human consumption is the principal product of many animal production industries, it is a further object of at least some embodiments of the present invention to not introduce any unacceptable contaminants, or unacceptable levels of contaminants, within such food products. Other objects of the invention will become clear upon a reading of the following specification.

AAHMS Key Features and Components

Several features and innovations of the present invention will help enhance utility of the disclosed AAHMS to the animal product production industry and to those responsible for other animal care environments, and also to consumers of animal products. These features relate to effectiveness, capabilities to easily tailor embodiments of the present invention to meet the specialized needs of different animal care environments, ease of use features to minimize direct labor and other direct costs required for installation and use of the AAHMS, and features to enable compliance with food safety regulations and other practices within the overall animal product production and other animal care environments.

Summary of Elements of Automated Animal Health Monitoring System (AAHMS)

Multiple alternative embodiments of the present invention are possible. Each embodiment would comprise some combination of some or all of the components illustrated in FIG. 3 and summarized as follows: wireless telesensor implants containing wireless telesensors 50, 51; programming, calibration, recharging, and/or activation units 52; squeeze chutes or other animal restraint devices modified to support installation of implants 54; installation tools 56; identification and/or relay tags 57, 58; special packaging for implant units to enhance efficiency of installation and ease of use in animal care environments involving many animals 60; wireless receivers, transmitters, transceivers, and/or transponders 62, 64, 66; data bases 68; central processing and control units 70; conventional computer networking capabilities 72; personnel alerting devices 74; data readout and/or programming units 76, 78; and ambient environment sensor units 80.

### BRIEF DESCRIPTION OF THE DRAWINGS

FIG. 1. Overview of Typical Cattle Feedlot Operation Illustrating Possible Placement of Some Elements of the AAHMS Invention

FIG. 2. Representative Destiny of Products from Cattle Feed Lot Operations

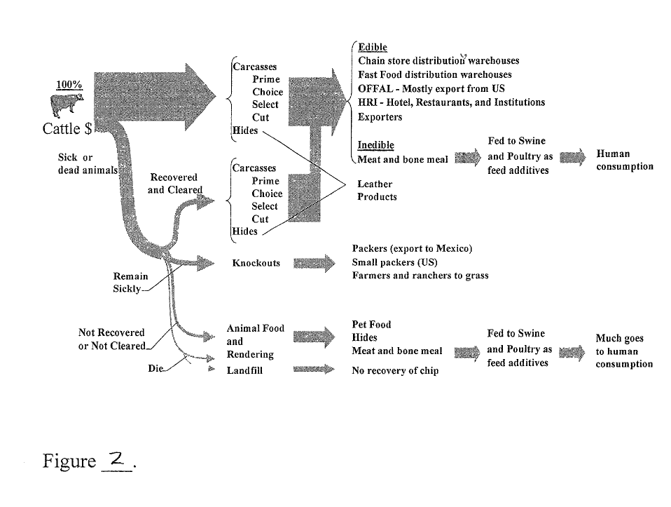

FIG. 3. Overview of Alternative AAHMS Elements and Interfaces

FIG. 4. Continuous remote ear-canal temperature record of a normal steer.

FIG. 6. A graph showing hourly temperature measurements of a steer.

FIG. 7. Skin and deep-body temperature changes of a steer following caretaker activity in the animal's room.

FIG. 8. Representative Top Level Logic Flow for AAHMS Telesensor Application

FIG. 9. Representative Logic Flow for Determining if Measured or Calculated Parameter(s) Is/Are in Bounds

FIG. 10. Representative Algorithm for Early Detection of Abnormal Condition

FIG. 11. Microcantilever Configuration for Shock or Vibration Sensing

FIG. 12. Wireless Temperature Sensor Block Diagram

FIG. 13. Wireless Temperature Sensor Block Diagram

FIG. 14. Ear Tag Containing, In Different Embodiments, Battery Storage Plus Either Transponder Style Relay, Receiver and Transmitter Relay (Half or Full Duplex), or Temperature Monitoring Circuitry Plus Transmitter Plus Optional Receiver

FIG. 15. Ear Tag Containing, In Alternate Embodiments, Solar Cell Power Generation and Battery Storage Plus Either Transponder Style Relay, Receiver and Transmitter Relay (Half or Full Duplex), or Temperature Monitoring Circuitry Plus Transmitter Plus Optional Receiver

FIG. 16. Representative Layout of Typical Implant Components

FIG. 17. Ear Canal Implant With Loops for Tool Similar to Snap Ring Pliers

FIG. 18. Ear Canal Implant With Tabs Which Can Be Grasped for Removal

FIG. 19. Ear Canal Implant With Wire Loop Which Can Be Grasped for Removal

FIG. 20. Representative Telesensor Implant Configurations Employing Plastic Battery Technology with Telesensor Electronics and Antenna Attached to or Sandwiched Between Sheets of Battery Material (Note holes through battery material to permit tissue to grow through and anchor implant.)

FIG. 21. Representative Telesensor Implant Configurations Employing Plastic Battery Technology with Telesensor Electronics and Antenna Attached to or Sandwiched Between Sheets of Battery Material, and with Surgical Mesh Material Attached to or Surrounding Implant to Promote Tissue In-growth Anchoring

FIG. 22. Representative Configuration of Telesensor Implant for Subcutaneous Installation, Showing Telesensor Electronics and Plastic Batteries, But with Antenna Extended and Coated with Collagen Sponge or Similar Material Which Promotes Tissue Ingrowth to Help Form a Biological Seal Where the Implant Penetrates the Skin or Hide.

FIG. 23. is a diagram of a steer head showing an implant location.

FIG. 24. Needle Implant Injection Gun With RF Readout and Bar Code Scanner.

FIG. 25. Representative Packaging of Implants

FIG. 26. is a configuration for a plastic battery.

FIG. 27. is an injection gun showing details of construction.

FIG. 28. shows configurations of cutting blades used in conjunction with an injection tool.

### DETAILED DESCRIPTION OF THE DRAWINGS

Principle of Operation

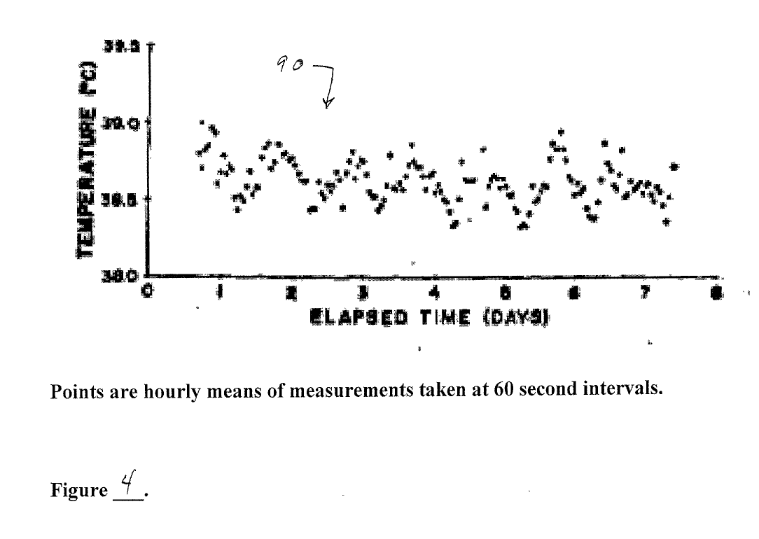

To be effective in the early detection and identification of sick animals, the AAHMS must be effective in monitoring parameters which relate directly or indirectly to the health and status of the animal. A mammal's body core temperature is one of the best indicators of their health and status. Most mammals have a measurable daily fluctuation in body core temperature about some “normal” value for their species, as illustrated in FIG. 4, which shows representative diurnal temperature variations for a healthy steer. In some cases, deviations from this normal temperature variations may be imposed by the animal's activity level, by changes in ambient conditions, or other influences, as illustrated in FIG. 5.

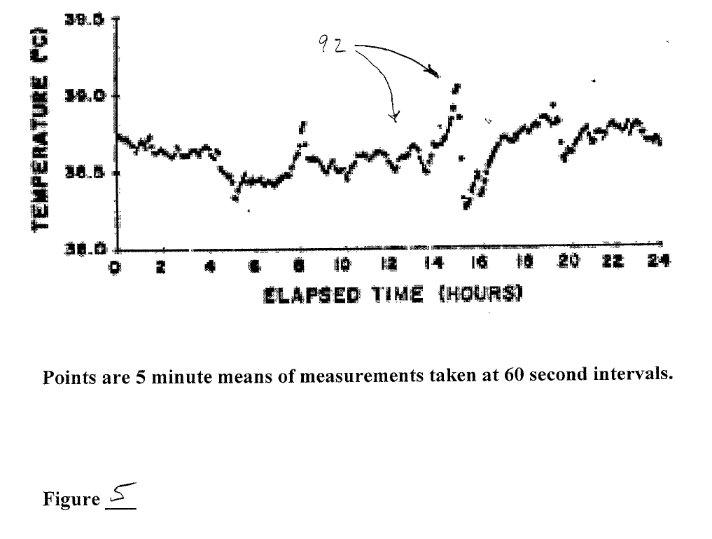

Temperature elevated 94 above normal diurnal fluctuations (i.e., fever) accompanies the onset of illness for most disease causing pathogens or conditions, as illustrated in FIG. 6. A mammal's depressed body core temperature (below the normal range) can indicate the onset of hypothermia or shock due to an injury, cold ambient environment, or other stress. From FIG. 6, one can also observe that, for at least some exposure levels of some pathogens, an animal's diurnal dip in temperature may be masked by the rate of rise 96 in temperature due the animal's response to one or more pathogens. One may also observe that the overall temperature response profile 94 of an animal vaccinated against the specific pathogen to which it is exposed is significantly different from the profile 98 of an animal which has not been so vaccinated. The temperature of the vaccinated animal increases 100 sooner after exposure to the pathogen, since the animal's immune system has already been sensitized for the specific pathogen by the vaccination. However, the temperature in the vaccinated animal does not go nearly as high as the temperature of the unvaccinated animal. Note also the sharp rise 96, 94 in temperature of the unvaccinated animal on the second day after exposure. Different pathogens may cause different temperature profiles. Having available the time history profile, over several hours or days, of the temperature or other health related parameter of an animal, as monitored by alternate embodiments the instant invention, may aid in diagnosis of an abnormal condition in the animal and also influence the selection of treatment options for the animal.

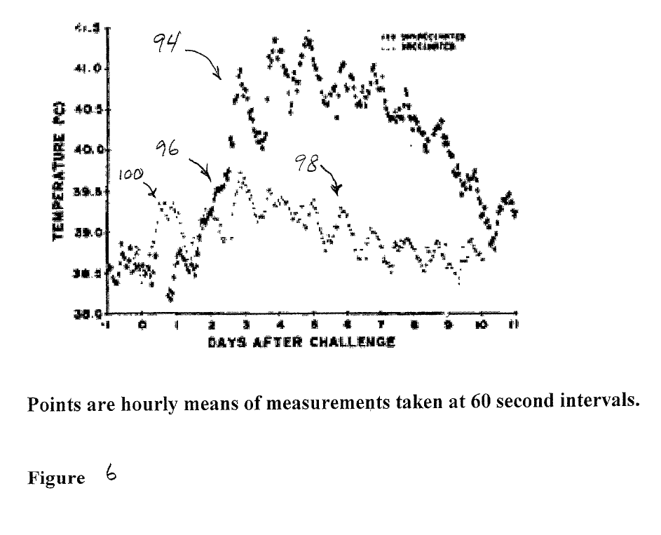

FIG. 7 provides an interesting observation regarding the relationship between an animal's skin temperature 110 and the animal's deep body temperature 112 in response to a mild stress. In this case, skin temperatures were monitored with a subdermally implanted transponder and deep body temperatures were monitored with an ear-canal thermister probe. The stress in this case was simply caretaker activity in the animal's room. A capability to monitor and compare the relationship (e.g., as a difference or a ratio) between an animal's deep body temperature and the animal's skin (subdermal) temperature can thus prove useful in monitoring for stress or shock in an animal. This capability, in turn, can be useful in monitoring animals for injury, entrapment, exposure to predators, or other conditions which could cause stress or danger to the animal.

There are other indicators of illness in an animal that may also be exploited for monitoring health and status of animals in different embodiments of an AAHMS. These include the fact that a sick animal will generally quit eating, may quit taking water, and will generally be more lethargic and less mobile in comparison to their companions in a feedlot or other animal care environment. Thus, a capability to monitor the mobility and changing locations within their environment (feedlot pen, pasture, corral, etc.) of animals being monitored, including whether they approach water and food sources, may also be a valuable indicator of an animal's health and status.

Another potentially exploitable indicator of the onset of respiratory diseases and other stressing conditions is coughing and certain body movements (e.g., rales or wheezing associated with onset of respiratory diseases, shivering associated with cold or stress, or shaking of the head associated with mad cow disease).

In some cases, it might be desirable to monitor for the onset of stress or mild shock that may be occasioned by various conditions, including entanglement or entrapment in fencing or other material, the approach of predatory animals, or even severe infestations of insects. The relationship of subdermal temperature to body core temperature (e.g., as a difference or a ratio) can be a useful indicator of stress or mild shock conditions in an animal (ref FIG. 7).

This relationship can be determined from measurements by two or more telesensor units with at least one being located just below the skin or hide, and the other unit being located in a position in the animal where body core temperature can be measured.

Another useful indicator of the onset of certain conditions affecting the health and status of animals, especially respiratory conditions such as pneumonia, is blood oxygen content.

Other useful indicators of the health and status of an animal include the respiratory rate, and the heartbeat rate (i.e., pulse rate), especially when coupled with knowledge of whether an animal is resting or moving about. Heartbeat rate may be sensed by any of several means, including, for example, electrical signals, acoustic signals and vibrations, and pressure waves and changes in blood density which may be sensed by blood oximetry as illustrated in a paper by T. L. Ferrell, et al (“Medical Telesensors,” SPIE Vol 3253, pp 193ff), incorporated herein by reference.

Electromagnetic signals generated by an animal's body may also be detected by appropriate sensors and exploited to monitor parameters related to the health and status of the animals, including, for example, heartbeat rate and other muscle activity, including muscle spasms induced by some diseases. Another useful indicator of the health and status is the white blood cell count. Blood pressure is yet another useful indicator of the health and status of an animal.

Another potentially exploitable indicator of the health and status of animals is the presence and concentration of pathogens, or other organisms or compounds associated with the presence of specific pathogens (e.g., antibodies), within the animals'lungs or blood, sputum, nasal drainage, breath, or other bodily fluids. A capability to directly monitor for pathogens or associated organisms or compounds would be a useful capability within an AAHMS, whether implemented to support broad surveillance monitoring or as a diagnostic capability after abnormal animals had been identified by other means.

Such a capability might be implemented, for example, employing an electronic biochip, such as that developed by Oak Ridge National Laboratory (ORNL) (and described in a paper by Vo-Dinh, T., et al, (Anal. Chem. 66, 1996; 3379-83) and which is incorporated herein by reference). This biochip is capable of detecting a DNA sequence or other signature components of specific pathogens. This biochip has been developed by the Health Sciences Research Division at ORNL to detect specific DNA targets. In material available publicly on the ORNL Internet Web site, and as described in the literature, it is indicated that the ORNL developed sensor will detect hybridized DNA without any external monitoring or signal transmission. The miniaturized device incorporates multiple biological sensing elements (i.e., DNA probes), excitation microlasers, a sampling waveguide equipped with optical detectors (fluorescence and Raman), integrated electro-optics, and a biotelemetric radio frequency signal generator.

Semiconductor microlaser arrays are incorporated into a device with oligonucleotides of specific DNA sequences attached to its surface. Free DNA sequences with fluorescent labels are allowed to hybridize (bind) to the oligonucleotides with which they have sequence homology. The fluorescently labeled DNA will emit light when it is illuminated at the optimum wavelength. The microlasers contained in the device illuminate each pixel on the surface, and the detector array identifies pixels that have fluorescent DNA sequences attached. A sensitive, accurate sensor has been demonstrated to identify, through their DNA, Pseudomonas organisms that are useful in bioremediation. Another sensor has been developed to identify the human p53 tumor suppressor gene. This technology may also be applied to identify other pathogens such as tuberculosis.

There are numerous other parameters and methods by which abnormal conditions in animals being monitored may be detected. The means identified above are intended to suggest the broad range of conditions and measurable parameters for which an AAHMS may be applied for automated monitoring of the health and status of animals in an animal production or animal care environment.

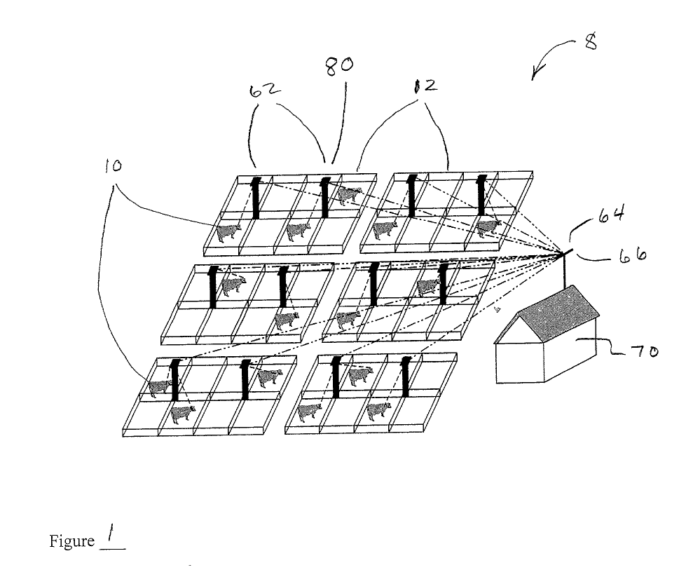

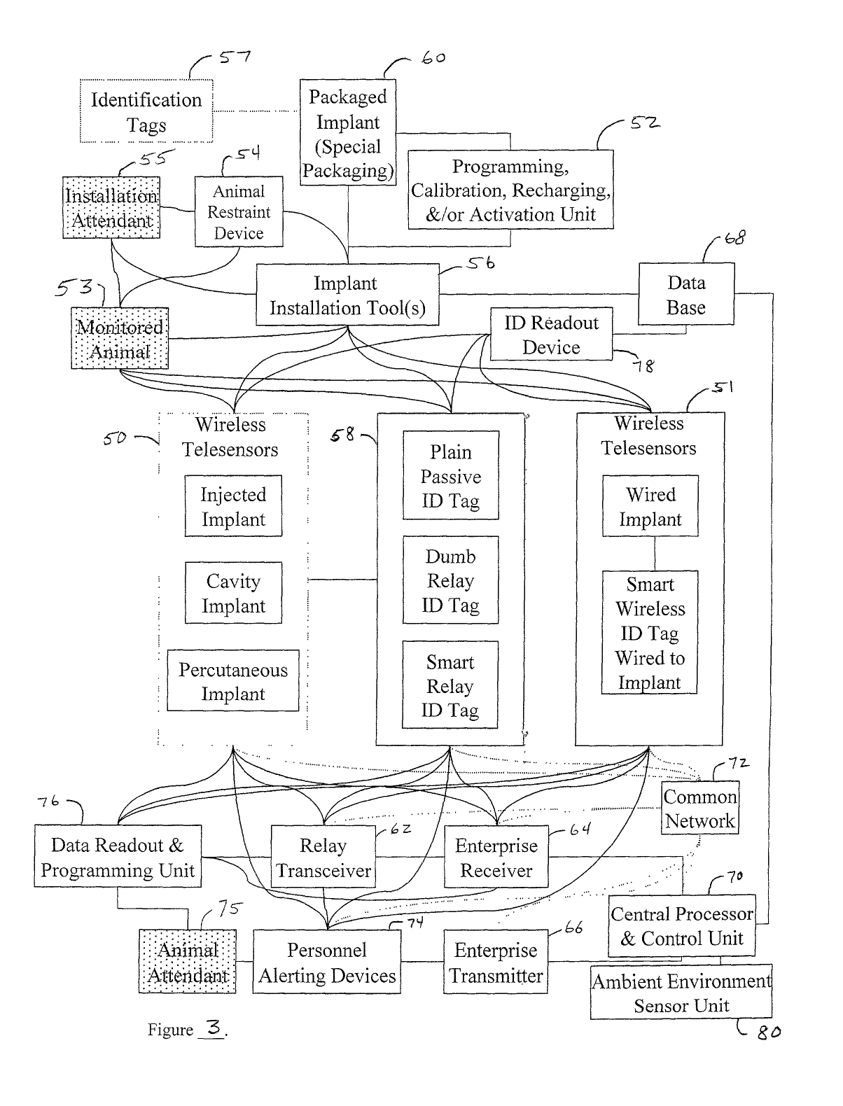

In a typical preferred embodiment of the present invention in an animal production or other animal care environment, such as the cattle feedlot environment 8 illustrated in FIG. 1, selected elements of the present invention are deployed along with smart telesensor implants installed in animals. Referring to FIG. 3, when an abnormal condition is detected by a smart telesensor 50, 51 (defined later herein), an alert is (1) transmitted directly to an alerting device 74 carried by or in the proximity of personnel responsible for animal care, or, (2) transmitted to one or more receivers 62, 64 and/or relay 58, 62 and/or processing devices 70 which ultimately transmits an alert including animal ID information, e.g., human readable ear tag number and, in some embodiments, animal location and/or other data, to personnel responsible for animal care via a personnel alerting device 74.

As indicated in FIG. 3, depending upon specific needs of different application environments, various economic tradeoffs, battery life versus size, installation depths within tissue, practical antenna lengths, RF frequencies employed, and other factors, the telesensor implants 50, 51 of the instant invention may be designed, in the same or different embodiments, to have sufficient transmission range to be received and relayed, or processed, by (1) receivers, transceivers, transponders, or other RF signal relay devices 58 mounted on or attached to the animal, directly or indirectly, by various devices (e.g., an ear tag, collar, belt, anklet), (2) receivers, transceivers, transponders, or RF signal relay devices in the vicinity of the pens, corals, stables, paddocks, pastures, open ranges or other environments in which the animals are maintained 62, (3) receivers 64 used in conjunction with other computers and information processing equipment to receive, process, store, and respond to information important to the functions and operations of the overall business enterprise, or (4) receivers used on shared or mutual access networks 72 to support other business enterprises (e.g., cellular phone networks, RF data networks, wireless access nodes for the Internet). Any specific embodiment of the instant invention may employ any or all of these intermediate methods of communications to transmit a warning signal from the telesensor implant 50, or a tag 51 or other device mounted on or attached to an animal, to a receiving device 74 (e.g., pager, PDA) carried or worn, in most embodiments and applications, by a person (e.g., attendant) responsible directly or indirectly for responding to alerts or warnings generated by the system for specific animals being monitored or for conditions threatening animals being monitored.

For those telesensor implants 50, 51 and application environments where the combination and tradeoffs of implant RF transmitter power, antenna length, RF signal attenuation and propagation distortion by passage through tissue, receiver antenna and receiver quality, and other factors result in an RF to signal too weak or too distorted to be detected at ranges convenient for economic installation of receivers in the vicinity of the cattle pens, an ear tag, surface mountable patch, collar, or other device attached to the animal and containing or supporting either a simple RF boost and relay capability 58, (e.g., a transponder), or a signal detection, processing, and transmission capability 58 may be employed to obtain the additional range and other functions as described hereinafter.

Integrated Use of AAHMS Elements to Maintain Identification Traceability

For a typical animal production environment or animal care environment where many animals are present, it is generally important that a unique identification (ID) code(s) in the telesensor implant(s) be correlated with a human readable identification code on the animal, preferably during installation of the telesensor implant(s) 50, 51. This is necessary so that when a telesensor implant 50, 51 transmits an alert or other signal from a sick or otherwise abnormal animal, the specific telesensor implant 50, 51 from which the alert is transmitted can be identified and associated with an animal in a way which positively identifies the animal in which the specific telesensor 50, 51 implant is installed. Thus, one of the important functions which the AAHMS must provide in most embodiments is the conversion of the electronic ID used to identify the telesensor implants 50, 51 installed in specific animals to the human readable symbols applied to tags 57, 58 or other devices.

As noted earlier, in some embodiments, the AAHMS may also provide a capability to determine the location of the animal from which a signal is transmitted.

The correlation of the telesensor ID code with the alphanumeric or other human readable symbols on a car tag or other device visible to humans can be accomplished in any of several different ways. Perhaps the simplest approach is to pre-package 60 ear tags 57, 58, 51, each containing a unique human readable ID code, together with corresponding telesensor implants 50, 51 which have been manufactured or pre-programmed with the same ID code so that the telesensors transmit an alphanumeric or other code directly corresponding with the human readable symbols and code on the ear tag 57, 58, 51. In this embodiment of this aspect of the instant invention, the installation attendant need only to insure that the ear tag 57, 58, 51 which came packaged with the telesensor implant is installed on the same animal in which the corresponding telesensor implant 50, 51 is installed. Alternatively, if the ID code in the telesensor 50, 51 and the ID code in the co-packaged 60 tag 57, 58, 51 are different, then a data file 68 and/or listing containing the appropriate correlations may also be delivered to the feedlot or other animal care environment to permit easy correlation of the codes. Since some feed lot operations already print their own ID tags 57, 58, a similar alternative is to provide the individual user locations with the tools 56 and devices 52 needed to enable them to print their own ID tags 57, 58 and program the telesensor implants 50, 51 with the same ID codes as printed on the tags 57 58, or maintain a record 68 of code correlations. Another related alternative is to use preprinted ID tags 57, 58 and provide the feed lot operators with the ability 52, 56 to program the same alphanumeric codes as used on the tags 57, 58, 51 into the telesensors 50, 51 as the telesensors 50, 51 are installed on the same animal. This may be performed conveniently by including a bar code or other machine readable code on the tags 57, 58, (whether preprinted or printed by the user). The bar code or other machine readable code may be read at the time of installation of the telesensor 50, 51 by a readout device integrated into the installation tool 56 (so the installation personnel do not have to waste time picking up another tool or device to effect the readout) or by a separate readout device appropriate (e.g., for passive electronic ID devices) for the type of machine readable code included on the ear tags 57, 58, 51. The telesensor implant 50, 51 can be programmed to the same ID code as used on the ID tag 57, 58, 51 by a programming unit 52 (including a capability built into the installation tool 56) such as described herein and employing an RF, IR, optical, acoustic or other receiver or special electrical contacts built into the installation tool 56 or by use of a separate programming and calibration unit 52, preferably located at the installation site to help insure that the telesensors 50, 51 programmed with ear tag ID codes are installed in the same animal as the ear tag 57, 58, 51 is installed on. Since more than one telesensor implant 50, 51 may be installed in the same animal, and for other reasons, it will generally be desirable to include a capability for automated readout and correlation, via a database 68 or other approach, of an ID code installed in each telesensor 50, 51 with the human readable ID code used on an ear tag 57, 58, 51 installed on the same animal.

It should be noted herein that the word “unique” as used herein to describe a telesensor 50, 51 or animal identification code is a relative term, and does not imply that certain codes never be repeated in a given application or embodiment of the instant invention. The principal goal of the use of such identification codes is to provide an efficient and unambiguous means of identifying the specific animal from which an alert is issued, and secondarily, to provide a means of identifying animals for routine record-keeping and management functions. Certain identification codes may be repeated within an overall AAHMS embodiment or implementation within a particular animal production or other animal care environment, especially when augmented by other procedural means to minimize the likelihood of confusion regarding the correlation of received alert warnings or data with the appropriate animal. Efforts are underway within the cattle industry to establish nationally or globally unique ID codes for animal ID and tracking from birth to death. If such a code is adopted, implants could be programmed with the unique ID code assigned to the animal being implanted.

During in-processing, individual animals are held relatively immobile in a squeeze chute or other restraining mechanism 54, generally including a restraint for the head, while the installation attendant 55 uses one or more implant tools 56 to insert one or more telesensor implants 50, 51 into the appropriate location(s) on the animal 53 (e.g., ear canal, or muscle and cartilage tissue just behind the ear attachment points).

If the arriving animal 53 already has an ear tag installed with a human readable identification code thereupon, but with no machine readable identification code (e.g., bar code, electronic ID chip with RF or IR readout, RF tag device), in-processing personnel 55 may elect to install a new tag as 57, 58, 51 described below, or, a human attendant 55 would record the human readable identification code from the existing ear tag manually on a paper log (for subsequent input to a data base 68) or by input to a computer (e.g., handheld, notebook, networked terminal) via keyboard, voice command and voice data entry, handwriting recognition (e.g., the Graffiti software employed on some palm sized computers) or other means.

In some cases, animals may arrive at the feed lot 8 (or other animal production or animal care environment) with ear tags or electronic ID devices already installed which contain a machine readable ID code (bar code, RF tag, microchip with ID code). In such cases, a capability to read out such ID codes may be added to the implant tool 56 of the instant invention, including a capability for generating the energizing RF electromagnetic fields where needed to power some types of electronic ID implants. Alternatively, a readout device 78 already designed for use with a particular type of ear tag identification code or electronic ID device may be used, but the device 78 would be modified if necessary to add a capability to transmit said ID code information to a computer data base 68, to the implant installation tool 56 of the instant invention, or to another device, where the machine readable ID code may be correlated with the human readable symbols on the ear tag or other type of tag attached to the animal to support efficient human identification of the animal from which telesensor 50, 51 generated alarm signals may arise.

As an alternative, or in addition to the above methods for maintaining correlation between the electronic ID code installed into the telesensor 50, 51 and visual identification of the animal from which a telesensor alarm signal originated, a digital image of the animal may also be captured during inprocessing with a digital camera (preferable networked via cable or wirelessly with the other in-processing tools and data base) and stored in a database 68 and correlated with the electronic ID code(s) of telesensor devices 50, 51 installed into the animal. For such purposes, in some embodiments, a digital camera could be integrated into a telesensor implant installation tool 56 of the instant invention, or a separate camera could be used, preferrably a digital camera which can be connected to a computer network and data base.

Without regard to whether ear tags or other means of identification are already affixed or installed in the animal 53 being in-processed, for ease of in-processing or for other reasons, a user 55 of the instant invention may elect to install an ear tag 57, 58, 51 as described later herein containing both human readable symbols and a machine readable ID code. In such cases, an installation attendant would use the optional ID readout capability integrated into the installation tool 56 (e.g., bar code scanner, RF tag reader), or a separate ID readout device 78, to read the machine readable ID code on the newly installed human readable tag 57, 58, 51 so that the machine readable code may be correlated with the human readable code and with the telesensor 50, 51 ID code in one or more databases 68 used to support identification of each animal 53 and, optionally, to maintain other information regarding each animal 53.

In a preferred embodiment, an implant installation tool 56 is used to either install and verify an identification code into the telesensor implant 50, 51, or read out an identification code already installed into the telensor implant 50, 51 during manufacture or by an optional implant programming and/or calibration unit 52. The said electronic ID code is transmitted via cable or wireless transmitter, either immediately or after input of the human readable ID code (as described below), to a computer hosting a database 68 which serves the purpose, among other things, of generating and/or maintaining records correlating the electronic ID code installed in the telesensor implant with the human readable ID code visible on a ear tag 57, 58, 51 or other tag affixed to the animal.

Again, several alternatives are available for correlating the ID code transmitted by the telesensor implant 50, 51 with the human readable ID code contained on an ear tag 57, 58, 51 or other human readable ID device installed on the animal. In most embodiments, the telesensor implant will transmit the ID code alone or with other data as part of the warning alert or alert warning notification to alert other elements of the AAHMS that an alert condition exists in a specific animal 53. In many embodiments, a central processing computer 70 may be used to correlate the transmitted ID code with the human readable code on the affected animal. Alternatively, hand-held or palm-sized computers or other such devices 74, 76 carried or worn by animal attendants 75 now have sufficient memory to contain the database needed for correlation for all animals on a large commercial feedlot. Many such commercial devices are now available, such as the Palm Pilots™ by 3-Com™ or Pocket PC devices such as the Hewlett Packard™ Jornado™ or the Casio™ Cassiopia™. RF and IR receiver cards are also available, or could be easily designed, for such devices which could enable them to receive alert warning signals directly from telesensors 50, 51 or from other transmitters 62, 66 employed in connection with the AAHMS, such as those indicated in FIG. 3.

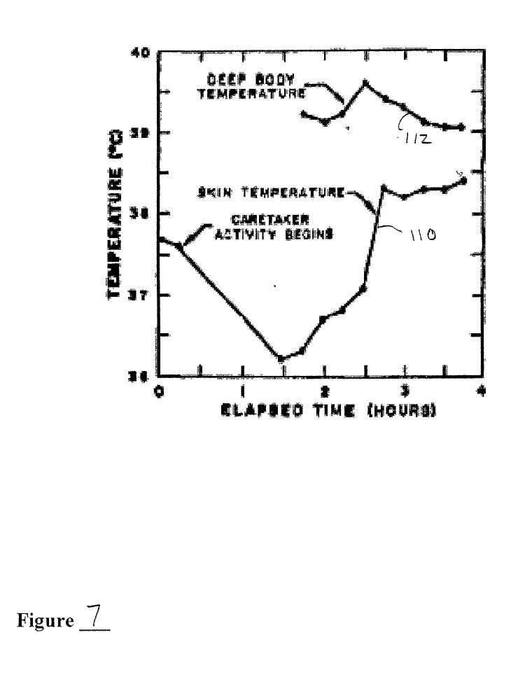

As noted earlier, a typical application of the AAHMS would be in monitoring the health and status of animals in a cattle feed lot operation 8. In such an embodiment, as an example, and referring to FIG. 8, telesensor implants 50, 51 of the instant invention (as described further later herein) capable of monitoring, or supporting monitoring, of one or more parameters or conditions related to the health and status of an animal would be made available 150 at the in-processing facility or location for newly arrived animals 154. Implants 50, 51 are activated and calibrated 152 as required, and identification codes and tags for animals 53 are read or installed 156 as required. One or more telesensor implants 50, 51 are installed in or on each animal 53 to be monitored. The implants 50, 51 may be installed 158 internally within tissue, externally within an ear canal or other open cavity, within closed cavities such as the vagina or rectum, or percutaneously, wherein most of the sensor implant is internal but a portion of the telesensor (normally only the antenna) penetrates the skin or hide of the animal. The telesensor implants 50, 51 may be programmed to transmit an alert and optionally data when certain conditions are met (as described later herein). Provisions (e.g., a database 68) are made to insure the installed telesensor is operational 160 and that the identification code transmitted by each telesensor implant 50, 51 can be correlated with a visible, human readable identification code also affixed to an animal 53 (e.g., on an ear tag 57, 58, 51).

After a telesensor implant 50, 51 is installed, it enters a monitoring cycle mode 162 and the animal is released or placed into an animal production or other animal care environment. In most preferred embodiments, a telesensor implant 50, 51 is designed so that sensor and processor related circuits, and, separately, RF transmitter related circuits, can be powered up or powered down separately under control of a controller circuit. Thus, in a typical feedlot animal monitoring application, the telesensor implant 50, 51 is programmed to be powered down to a standby state most of the time. In many embodiments, a trigger mechanism (e.g., RF or FR receive circuits) is provided to permit a user with appropriate equipment (e.g., a data readout and programming unit 76) to request a readout 164. Normally, a time-keeping circuit is employed in the standby state to permit the telesensor implant to be programmed to power up at least those circuits supporting sensor functions at certain preprogrammed intervals 166 or upon a particular schedule. For such embodiments, at each power-up cycle 168, the telesensor will typically make one or more measurements 170 of one or more parameters (e.g., temperature, blood oxygen). In many preferred embodiments, the controller portion of the telesensor may then store 170 the measurement data, including, in some cases, time of the measurement, and may also employ 172 one or more criteria or algorithms, examples of which are indicated in FIGS. 8, 9, and 10 to determine if sensor data and/or an alert should be transmitted. When criteria for transmission of an alert or data are met, the telesensor may be programmed to enter an alert transmission cycle 178 during which it will power up additional circuits 180 associated with the transmitter and transmit 182 the alert or data. The telesensor will then typically power back down to a standby state to minimize power usage and thereby enhance battery life 184. In the illustrated embodiment, the telesensor waits a prescribed time 190, and if an acknowledgment 186 or readout request 188 is not received, activates another transmission 180-184. If parameters are normal and no alert is needed 172, the telesensor schedules 174 the next measurement and powers down 176 to standby mode to await the next measurement. If an acknowledgment was received 186, and a readout was requested 188, the telesensor activates 192 the transmitter circuits, transmits data 194, powers down 196 the transmitter, schedules 198 the next measurement, and returns 176 to standby

Most parameters related to health and status are sensed directly by the telesensor implant. Other parameters, particularly the animal's location and movement, may be monitored or derived by other sensors 80, receivers 62, or processors 70 operating in conjunction with the telesensor (as described later herein).

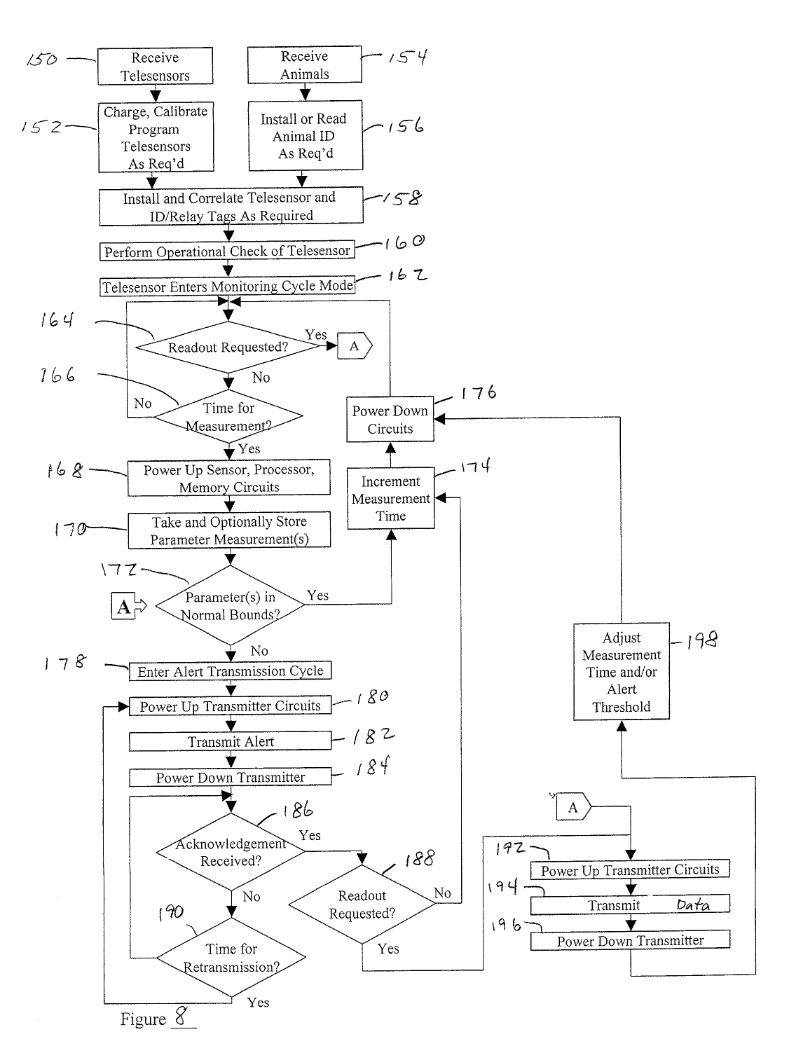

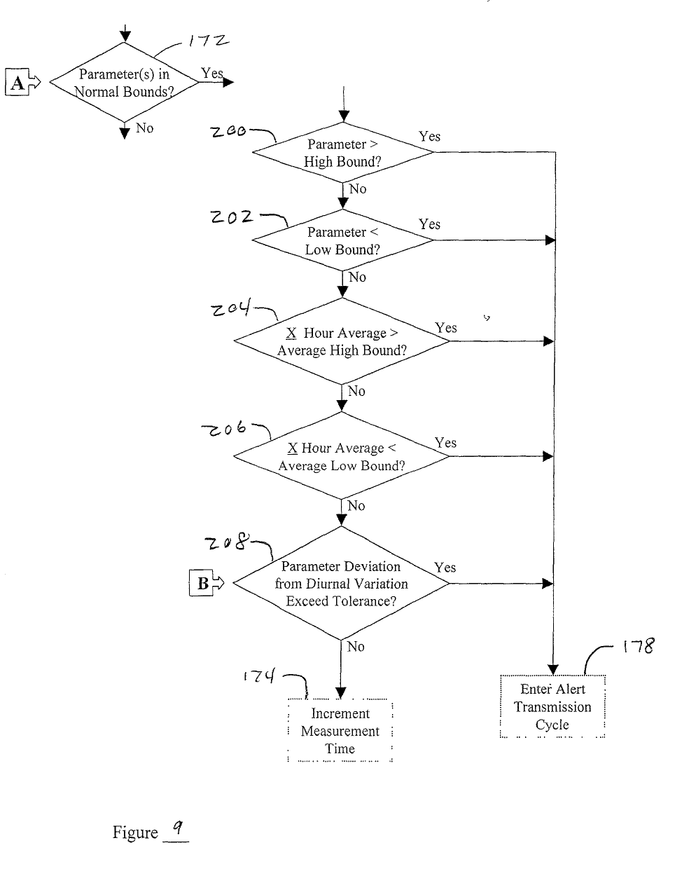

FIG. 9 may be viewed as an expansion of block 172 in FIG. 8, and provides an example of some criteria which could be implemented to determine if an alert should be transmitted. For example, at each measurement cycle, the telesensor may check temperature or some other parameter to determine 200 if a high threshold was exceeded. If not, the telesensor could check 202 whether the measured value was lower than a low threshold for the parameter. If not, the telesensor, in some embodiments, would retrieve previous measured values from memory to determine 204 if the average of the measured values over some number of hours X, for example 4 hours, exceeds some average high bound (selected to achieve a balance between false alarms and earlier detection of the onset of illness). If that criteria was not met, an additional similar check 206 could be made to determine if the average of the measurements over some period was lower than some average low bound. If not, additional, more sophisticated checks could be made, as indicated at block 208. An example of such an additional check is illustrated in FIG. 10, discussed below. Continuing with FIG. 9. If no alert condition was detected, in this embodiment, the telesensor would schedule 174 the next measurement and continue as shown previously in FIG. 8. If telesensor processing detected an alert condition, the telesensor would proceed to enter 178 on alert transmission cycle, as illustrated earlier in FIG. 8.

For a cattle feedlot operation, a typical readout cycle may consist of transmitting three (3) alert warnings at random intervals between one (1) and ten (10) seconds, waiting fifteen minutes for an attendant with a readout unit, or another transmitter within the AAHMS, to respond, then repeating the transmission of three (3) warnings again as noted above, then waiting again for fifteen minutes for a response. For telesensor embodiments without a receiver, this alert warning cycle is typically be repeated only a few times to conserve battery life, after which appropriate circuits would be powered down to await the next programmed measurement interval. In telesensor embodiments with a receiver or direct input capability, if only an acknowledgement is received by the telesensor, the affected telesensor, typically, simply increments the time for the next measurement, powers down circuits as appropriate, and awaits the next programmed measurement time. For cases where an additional data readout is requested remotely by the system or by a data readout unit being used by animal attendent personnel, the telesensor unit simply powers up the readout circuits and transmits the requested data, then adjusts the time for the next measurement, and powers back down to await the next measurement time. Typically, for telesensor embodiments containing a receiver or direct input capability, measurements or readouts of stored data may also be requested at any time, as also indicated in FIG. 8.

FIG. 9 indicates some of the criteria which may be programmed into telesensor units in certain embodiments to enable the telesensor unit to determine when a warning alert should be transmitted. Data acquired by initial use of an AAHMS embodiment or prototype in a particular animal production or other animal care environment can be used to adjust the alarm thresholds and other control parameters and algorithms to provide the desired balance between early warning and false alarm rate. Adjustments may be tailored to different types of animals, including different breeds of the same animal, different environments (e.g., indoor, outdoor, seasonal), different diets or other conditions to achieve the desired warning or data readout response for a particular application and embodiment of the AAHMS.

FIG. 10 illustrates an example algorithm which could be used to determine when the temperature or some other health and status related parameter deviates by more than a programmable threshold amount from the normal diurnal variation. This figure may be viewed as one candidate algorithm that could be implemented in block 208 of FIG. 9. In this example, the diurnal variation of a particular parameter, such as temperature, may be represented to some approximation by a sine wave (see FIG. 4). A test 210 is implemented to determine if a newly measured value deviates from the expected value for that time of day by more than some acceptable tolerance amount. Programmable control parameters permit adjustments for the normal amplitude and phase (based on time of day) of the sinusoidal variation to align a particular measurement at a particular time with the corresponding expected value of the normal sinusoidal variation. If the measurement value deviates from the expected value by more than some tolerance amount, conditions for an alarm warning would be met and the telesensor would initiate alert transmission cycle 178. The tolerance value may be a constant or may itself be a function of the time or day or the phase of the diurnal variation. The value(s) for the tolerance may be input as a constant, an array of values related to time or day or phase of the cycle, or a function having other values that may be adjusted. If the alert criteria is not met, the telesensor would schedule 174 the next measurement and power down as shown in FIG. 8. The use of a sine wave to model the expected diurnal variations is only an example. Many other techniques and algorithms may also be used to enable a telesensor to determine when a particular parameter being monitored has deviated sufficiently from its expected value to warrant issuance of an alert warning.

In telesensors capable of monitoring for more than one parameter, or more than one abnormal condition of a parameters (e.g., hyperthermia vs hypothermia), or the degree of variation of measured versus normal parameter values an additional code or data may be included in the warning alert transmission to permit a determination by the AAHMS or responsible personnel of the urgency of the response.

In addition to conserving energy, another reason the transmitter circuits are powered up only when needed is that the heat generated and dissipated from the transmitter portions of the micro-chips while the transmitter circuitry is operational may cause a temperature rise in the micro-chip which may interfere with temperature measurements and possibly measurements of other parameters being monitored in some embodiments of the instant invention.

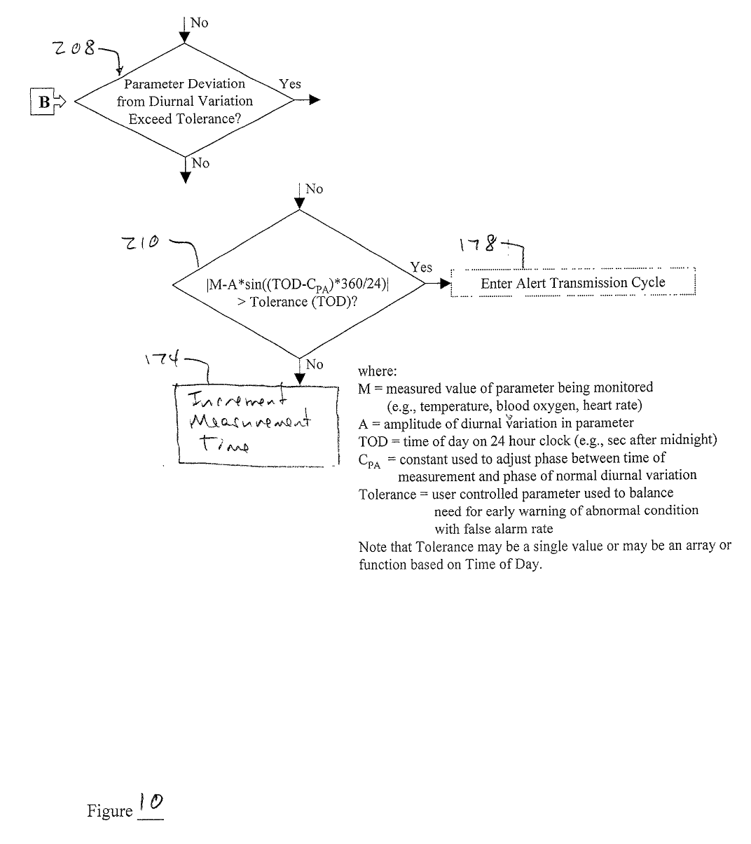

In some embodiments, one or more sensor circuits may remain active during the power down states. In other embodiments, the telesensor may then be programmed to power up when certain sensor input criteria (i.e., trigger criteria) are met. For example, a telesensor may employ one or more miniature bimetal thermostats to close and activate the implant when a temperature threshold (high or low) is met. Alternatively, a telesensor may employ one or more micro-cantilevers 250, such as those used in atomic force microscopes, with small masses 252 near the tips of the micro-cantilever arms 262, as illustrated in FIG. 11. The micro-cantilevers may be oriented in different directions so as to provide three-axis detection of shocks, vibrations, or other accelerations. In a preferred embodiment, the micro-cantilever shock and vibration sensors are assembled with a layer of piezo-electric material 254 as indicated in FIG. 11 capable of generating a voltage output more or less proportional to the amount of deflection of the micro-cantilever. (Although many other alternative readout techniques for micro-cantilevers are possible and well understood, see Sarid, 1991, piezo-electric and similar techniques may require less electrical energy to implement, and hence prolong battery life where important). The micro-cantilevers in this embodiment would function as accelerometers and be capable of providing a measure of the accelerations experienced by the animal. The response frequencies (i.e., resonant frequencies) of the micro-cantilevers 250 can be modified by varying the mass 252, the length and stiffness of the micro-cantilever, and other parameters. For example, if the telesensor containing such micro-cantilevers were mounted in or on the head of the animal, the sensitivity threshold of the detector circuits could be adjusted to respond to a certain level of acceleration which may correspond to accelerations induced, for example, by coughing or by shaking of the head (a characteristic of mad cow disease). In such an embodiment, as an example, upon being triggered by a voltage output from the piezo-electric layer in a micro-cantilever which exceeds a certain threshold, the telesensor may be programmed to wake up, record and store the time when it was triggered (and optionally other data), apply an algorithm to use previously stored times from previous trigger events to determine how many times the acceleration threshold has been exceeded (i.e., a trigger event) over some time interval (e.g., a measure of how often the animal is coughing). The sensor may then transmit an alert if a certain number of trigger events is exceeded over a given time interval so the animal may be checked out by an attendant. Similarly, the resonance response frequencies of the micro-cantilever may be adjusted to frequencies which would be excited by the raspy, wheezing breathing associated with many respiratory diseases. An analog or digital filter (preferably analog to conserve power) could be employed on the telesensor input, along with an integration circuit, to provide a sensor that selectively responds to the aforementioned raspy or wheezing breathing. The telesensor arm 262 is supported from a block 256 mounted on a substrate 258. A restraining member 260 is used to prevent damage to the microcantilever if subjected to a high g-load.

For temperature sensing applications a miniature bimetal strip thermostat can be employed. Alternatively, a microcantilever configuration can be configured with two materials having different thermal expansion coefficients, thereby having a deflection characteristic which is a function of temperature, similar to the response of a bimetal strip thermostat. Miniature contacts can be used to activate other circuitry and functions in a telesensor unit.

When criteria such as those mentioned above are met and the telesensor transmits an alert, the transmission may consist of simply transmitting the ID code installed in the particular telesensor. Alternatively, separate or additional data may be transmitted. The transmission may use any of many different encoding and modulation techniques and RF carrier frequencies compatible with the field of application and frequency usage and licensing regulations application to the geographic area in which any particular embodiment of the present invention may be used. There are multiple tradeoffs which can be made by those skilled in the arts of radio communications and electronics regarding carrier frequency, antenna lengths and sizes, tissue attenuation, regulatory requirements (e.g., FCC rules and international treaties related to frequency allocation and usage), propagation characteristics, transmitted waveform, and technology complexity and maturity.

Depending upon the number of animals and telesensors in a given area within range of one or more receivers, there is a possibility of two or more transmitters transmitting at the same time. When the telesensors are used in their preferred mode of transmitting only when an abnormal condition is detected, the probability of signals from two or more telesensors arriving at a receiver at the same time will be low for most applications. However, in order to minimize the possibility that alert warning signals from telesensors transmitted at approximately the same time may overlap in time at the receiver and not be properly detected, provisions should be made in most embodiments for multiple transmissions of alert warning signals from any given telesensor when an alert condition is detected. Random access schemes such as the Aloha access scheme (see Information Disclosure Sheet) have been developed for such conditions. In such schemes, each transmitter attempting to be “heard” makes multiple transmissions, but the delays between the transmissions are typically based on independent random draws by each transmitter over some time window. With such a scheme, even if the signals from two or more telesensors arrive at the receiver at the same time and interfere destructively at one interval of time, the independent random draws should virtually insure that when their next transmission attempts are made, they will transmit at sufficiently different times so that they do not interfere. For most preferred implementations involving smart telesensors, this approach should suffice. For implementations of the instant invention involving many animals and frequent transmission of data from telesensors in each animal, a more sophisticated multiple access scheme may be required. Several other network resource access schemes have been developed and documented in the literature and are well understood by those skilled in the art of digital communications. Several sources containing such information are listed in the information disclosure document associated with this application.

Some implementations of the instant invention may employ a spread spectrum waveform, such as a direct sequence spread spectrum (DSSS) waveform. Spread spectrum communications links properly implemented by those skilled in the art, especially those incorporating forward error correction encoding and interleaving, offer a tradeoff between signal processing and transmitter power with the advantage that a communication link can generally be implemented with less transmitter power, particularly in links where signal fading from multipath reflections and other propagation anomalies are present.

In spread spectrum implementations, the spreading waveform may be implemented with orthogonal code sequences, such as a Gold code sequence, with sufficient code length to provide a number of unique code sequences. Spread spectrum transmissions implemented with such waveforms may be independently detected by a properly configured spread spectrum receiver even though they arrive overlapped in time, since the receiver can use independent replicas of the orthogonal spreading code sequence to despread the signal. The orthogonal properties of the spreading code in effect create separate code channels and permit simultaneous reception of information from two or more transmitters in a properly configured receiver employing multiple receive channels. This technique is referred to in many texts and handbooks as Code Division Multiple Access (CDMA) (See Information Disclosure Sheet). The level of Very Large Scale Integration (VLSI) now achievable in micro-circuit design and implementation now permits such a capability to be implemented within micro-chips which can be mass produced at low cost per chip. As described later herein, Oak Ridge National Laboratory (ORNL) has announced that they have developed and prototyped a VLSI implementation of a miniature spread spectrum transmitter, along with sensor circuits, on a micro-chip approximately 3 mm by 3 mm by 1 mm in dimensions. The Gold code used in their early prototypes provides approximately 63 independent spreading sequences. With a properly designed receiver and system concept, this would permit simultaneous or overlapping transmissions from multiple telesensors deployed within range of a given receiver to be detected.

For some applications, it may be desirable to select the length of the orthogonal spreading code such that the number of unique code sequences exceeds the number of telesensors which would be within range of the spread spectrum receiver at any one time. In such a case, the transmitter may simply transmit multiple cycles of the spreading waveform without imposing any additional data modulation for identification. The spread spectrum receiver can determine which telesensor transmitted the alert by determining which code sequence despread the code with the highest correlator output. This would provide an alternative means for electronic identification compared with simply transmitting a unique ID as alphanumeric data.

Spread spectrum waveforms offer still another capability, namely the capability to precisely determine the location of the transmitter, a feature useful for embodiments of the instant invention. Since the code correlation procedure for despreading the spread spectrum signal can provide a precise and unambiguous relative time-of-arrival reference between multiple receivers of the same transmitted signal, multiple time synchronized receivers can be employed along with triangulation to determine the location of the transmitting telesensor installed in an animal. With proper selection of the spreading waveform and provisions for transferring synchronization signals between multiple receivers deployed around the general location of the animals being monitored, it is practical to determine the location of the telesensor, and consequently the animal, to within a few feet. The techniques for this radiolocation feature are similar to those used in the Global Positioning System and are familiar to those skilled in the art.

For telesensors employing RF receive capability, a range interrogation feature, similar to radar transponders used in commercial airliners and air traffic radars, can be added to aid in radiolocation of a telesensor with fewer receiver sites in the animal care environment. As noted earlier, this capability will be useful in some embodiments of the instant invention to help determine whether the animal is moving about normally, and to help responding personnel locate the specific animal from which an alert warning was transmitted in a pen or pasture containing many animals.

The radio-location feature described above which can be implemented in certain embodiments of the instant invention will require a transmission by the telesensor each time a position reference is desired. Consequently, the use of this feature for frequent monitoring of an animal's location may significantly reduce battery live and should involve appropriate tradeoffs compared with the benefits of position monitoring. In embodiments of the instant invention wherein a receive capability is built into the telesensor chip so that additional transmissions can be commanded by remote transmitters, this radio-location feature is particularly useful to enable enhanced correlation and analyses of observations of different parameters to determine when an abnormal condition exists requiring the attention of attendants. For example, if a deviation from the normal diurnal variation in temperature is detected by an algorithm such as that described in FIG. 10, multiple interrogations of the animal's position may be made by the mechanism described above to determine, for example, whether the cause of the elevated temperature may be due to the animal running or otherwise moving about. In embodiments involving data bases 68 and ambient environment monitors 80 as described elsewhere herein, correlations and analyses may also be made with the solar radiance and ambient temperature, and the solar absorption characteristic of the particular animal—e.g., black angus vs a lighter colored breed—to determine whether a given temperature fluctuation was normal or requires response by appropriate personnel.

For embodiments of the instant invention where frequent determination of the animal's location is required, global positioning system (GPS) and/or differential GPS receivers can be implemented in micro-chips and added, for example, to ear tags, collars, or other devices attached to animals being monitored for health and status or for other purposes. The GPS or differential GPS receivers will permit determination of the location of the animal without requiring energy consuming transmissions from a temperature telesensor implant. The GPS derived location information can be provided via wired or wireless connection to telesensors of the instant invention, also installed on or attached to the animals. Algorithms implemented within the telesensor micro-controller may then determine when an abnormal condition exists (e.g., no significant movement over a period of several hours at a certain time of day and transmit a warning alert.), or upon a scheduled interval, a command, or other criteria, transmit the animal's location or other data to the receivers of the instant invention, such as illustrated in FIG. 3. As noted herein, position data may also be integrated with other algorithms employing, for example, pulse rate, to determine when an abnormal condition exists requiring transmission of an alert or other actions by the telesensor or the overall AAHS system.

Referring back to FIG. 8, once an abnormal condition is detected and conditions requiring transmission of an alert warning are met, the telesensor typically enters an alert transmission cycle. Upon entering an alert transmission cycle, the controller portion of the telesensor powers up the transmitter portion of the telesensor and transmits the alert warning, and, in some embodiments, additional data multiple times with random delays between each transmission. The telesensor controller then removes power from the transmitter circuits to conserve battery life, and uses time-keeping circuits to wait an appropriate time (normally preprogrammed) for the next alert warning transmission. In embodiments wherein an RF, optical, or acoustic receiver, or a direct input capability (e.g., wired contacts, pushbutton) has been integrated into the telesensor, the receiver is activated and listens for an acknowledgment or command from a data readout and programming unit or from another transmitter in the overall AAHMS.

Wireless Telesensors

A wireless telesensor unit 50, 51 (implant or external), or, simply, telesensor, as also referred to herein, is a device created by the integration of one or more micro-chips, a power source (e.g., battery, solar cell), an antenna, and, in some embodiments, additional sensors or other components which together provide a capability for measuring one or more parameters and/or transmitting the data or a warning or identification signal (e.g., a unique ID code associated with the telesensor) via RF and/or IR signals. Most embodiments of the instant invention employ wireless telesensor implant units 50 installed internal to the animals to be monitored, or wireless telesensor external units 51 attached to said animals as, or on, an ear tag, collar, adhesive skin patch, or other attachment devices, and, in some embodiments, employing sensors installed in cavities in the animal such as the ear canal and connected by wire to telesensor electronics installed in said tag, collar, patch, or other attachment devices. Some embodiments employ telesensor implant units designed to be installed in an ear canal or other cavity (e.g., vagina, rectum) and employing battery powered wireless transmitters to transmit identification or other data to receivers located on the animal or in the vicinity of the animal. Some embodiments employ percutaneous implants which are installed in the animals to be monitored in such a manner that a portion of the implant is within the animal and a portion of the implant (normally only the antenna) penetrates the skin of the animal.

The telesensors (implants or external units) of the instant invention include one or more electronic microchips, at least one of which has a built-in micro-controller capable of controlling the sequencing and performance of other functions built into the micro-chips. Some of the micro-chips employed in some embodiments of the instant invention are capable of measuring, or supporting measurements of, parameters (e.g., temperature, blood oxygen) related to the health and status of animals by employing built-in sensors, external (to the micro-chip) sensors, or a combination of internal and external sensors.

Some of the telesensor units employed in the instant invention may contain RF transmitters; some may also contain RF receivers or transceivers; some may contain infrared (IR) receivers, IR transmitters, or IR transceivers; and some may contain an acoustic receiver or other signal reception capability for commands and other data, including direct wire contacts and/or buttons and switches. The RF transmitters, RF receivers or RF transceivers, may include, in some cases, capabilities to transmit and/or receive a spread-spectrum waveform.

In some embodiments the telesensor units may also contain digital memory or otherwise provide for the storage of digital information. Some of the micro-chips may also contain processing and logic capability which permit the execution of certain algorithms to help control the operations of the implant and/or to help determine when an abnormal condition exists related to the health and status of the animal being monitored.

Depending upon additional capabilities also integrated into or with the telesensors of the instant invention, the telesensors may also be categorized for discussion herein as “dumb” or “smart.” In our terminology, dumb telesensors contain the capability to make and transmit one or more measurements at predefined intervals, or when certain conditions are met, and may contain a capability to selectively power-up or power-down certain other functions of the micro-chip or micro-chips contained in the telesensor unit, but dumb telesensors lack the ability to process data from multiple measurements to determine when an abnormal condition exists, or to control additional measurements based upon the findings of previous measurements.

In our terminology, smart telesensors have some or all of the functionality described above for dumb telesensors, but also have the storage and processing capability needed to implement one or more algorithms requiring memory of previous measurements to determine whether criteria requiring transmission of a warning alert or measurement data have been met. Smart telesensors may also employ the same or additional algorithms and logic to modify and selectively implement additional functions of the telesensor unit (e.g., making additional measurements or entering an alert transmission cycle, including powering up the transmission circuits).

It should be noted here that one key reason for integrating additional storage and processing capability into a telesensor (to create a smart vs dumb telesensor) is to help conserve battery energy, which may be a limiting constraint in some embodiments where both small size and long term operations without a requirement for recharging are desirable features. Generally, transmissions via RF or IR of data or warning alerts are among the most energy consuming functions of the telesensors as described herein. Thus, a dumb telesensor which includes transmission of data at multiple measurement times will generally consume much more energy, and consequently lead to shorter battery life, than a smart telesensor which makes, stores, and analyzes sensor measurements at each measurement interval, and then powers-up the RF or IR transmission circuits to make a transmission only when certain criteria are met. A smart telesensor may operate in an animal and provide effective monitoring for weeks or months without making an energy consuming transmission. Nevertheless, some applications and users will require more frequent transmission of measured parameters. Consequently, alternate embodiments of the instant invention provide for use of either dumb or smart telesensors.

Thus, the telesensor element(s) of embodiments of the instant invention comprises some or all of the following:

dumb or smart, battery-powered, wireless telesensors, as described further herein, integrated into a swallowable capsule or implants for the ear canal, vaginal canal, rectum, throat, or nostril;

dumb or smart, battery powered, wireless telesensors, as described further herein, integrated into subcutaneous or percutaneous implants injectable by needle or similar device as described herein into animal tissue or implants with an outer form resembling a screw-in wall anchor which could be installed in an animal with a screw-like action;

dumb or smart, battery powered, wireless telesensors, as described above, integrated into a skin penetration and retention apparatus (called “percutaneous implant” herein) wherein a portion of the implant (normally only the antenna) penetrates the skin or hide of the animal and is exposed outside the animal, as described further herein;

dumb or smart, battery powered, wireless telesensors, as described above, integrated into an ear tag, collar, or other animal attachment device, and including, in alternate embodiments, a wired connection to a temperature sensor (e.g., thermistor, thermocouple), blood oxygen sensor, heart rate sensor, or other sensing element implanted into the ear canal of the animal being monitored. In such embodiments the ear tag or other external attachment device may also display a human readable identification code, and may also comprise one or more batteries including, in some embodiments, plastic batteries, and, in some embodiments, solar cells to provide power for the telesensor.

Telesensor Implants

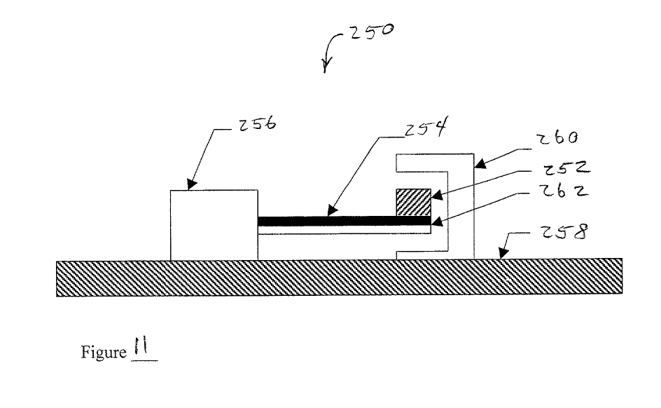

The wireless telesensor implants of the instant invention are key components in most embodiments of the instant invention. Referring to FIG. 12 as an example, the telesensor implants 280 are generally comprised of one or more electronic micro-chips including a micro-controller 282, reference oscillator 284, memory 286, along with sensors 292, an antenna, and a battery 288 which provide sensing, control, power management, data storage, and transmission functions. The telesensor implant 280 also includes supporting 294 and sealing materials 290 and special coatings 296 and other components needed to help provide the physical interface with the body of the animal being monitored, and to support installation or removal or recovery or reuse of the telesensor implant.

The electronic micro-chips and other components may also provide, in some embodiments, a reception capability for RF, optical (including infrared), acoustic, or other signals, and may also provide for reception of input data and commands from wired contacts or from built-in switches or other controls. This reception capability permits commands, control parameters and other data, and algorithms to be transmitted to the micro-chips and other electronics contained within the telesensor implant either before installation into or on the animals to be monitored, while installed in or on the animals, or at other times.

In most embodiments, the telesensor implants 280 of the instant invention contain batteries 288 or other energy generation or energy storage devices, such as low leakage capacitors, to permit storage of energy and operation of the telesensor electronics without a requirement for activation by an external power source (e.g., via electromagnetic fields induced by hand-held wands) to provide power for a measurement or readout at the time of the measurement or readout. (This statement is intended to distinguish the telesensors of the instant invention from other electronic implants which require activation by an external energy source at the time of the measurement and readout, but is not intended to preclude the use of appropriate features and circuitry to permit recharging of the battery or batteries contained on or within the implant, either while the telesensor implants are installed in or on the animal, or upon removal from the animal or to preclude use of an energizing RF coil to supplement or replace battery power for some operational modes.) In different embodiments of the implants and telesensor electronics, one or more different sensor capabilities may be integrated with the implant, including, for example, temperature sensors, blood oxygen sensors, and shock/vibration/acceleration sensors with sensitivities adjusted to different operational regimes as appropriate for the signal or condition being sensed.

Telesensor Electronic Micro-chips

The telesensor electronic micro-chips used within the instant invention are based upon large scale integrated (LSI) and very large scale integrated (VLSI) micro-circuit technology which enables the innovative integration of key functions needed to support implementation of telesensor implants of different embodiments of the instant invention into a single or a few small electronic chips which require only small amounts of power to operate. These key functions include measurement, data conditioning, storage, processing, transmission, reception, power-management, and other control functions.

In addition, advances in battery technology can provide enhanced energy densities and battery life, and some battery technologies (e.g., plastic battery technologies such as developed by Johns Hopkins University researchers, see appendix B) provide useful energy densities with low toxicity of the material and chemicals used within the batteries.

Those skilled in the arts of electronic micro-circuit and application specific integrated circuit design and fabrication are currently working in normal industry practice with design rules of 0.5 microns and below. This enables the integration of significant functionality into a single or few electronic micro-chips which require little power and which can be mass produced at low production cost per unit.

Oak Ridge National Laboratory (ORNL) announced in the Commerce Business Daily (CBD) dated Apr. 23, 1999 that they had developed a wireless spread-spectrum temperature telemetry system on a single silicon chip and requested expression of interest from companies interested in licensing and commercializing their wireless sensor technologies.

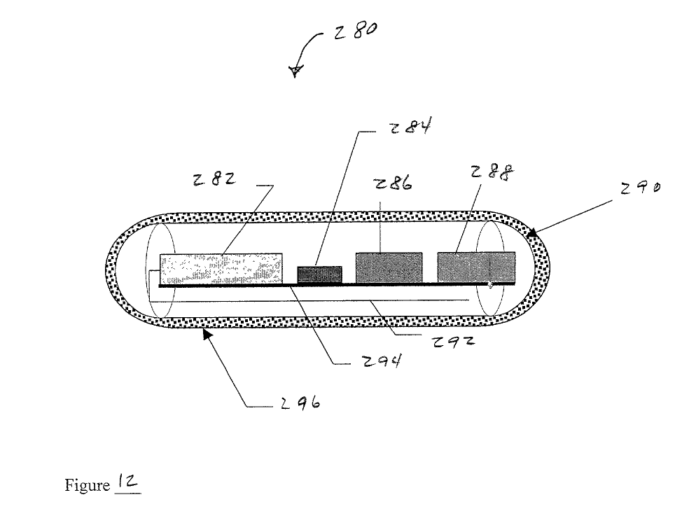

The above literature provided by ORNL, and which is incorporated herein by reference, described a Multichannel Integrated Spread-spectrum Telesensor (MIST) electronic microchip. The ORNL literature describes the chip as the world's first fully integrated spread-spectrum wireless temperature telemetry system on a single silicon chip. Referring to FIG. 13, incorporated into the chip 300 are: wide-range electronic thermistor 302, 304; two external sensor inputs 306, 308; a 4-channel, 10-bit data digitizer 310, 312; and a programmable direct-sequence spread-spectrum radio transmitter 318, 320, 322, 324 for use in the license-free 915 MHz RF band. The low-power, battery-operated device performs automatic, unattended sampling of local temperature 302, 304 and two external sensors 306, 308 and digitally reports the data via highly robust periodic spread-spectrum bursts up to 100 feet to a group receiver. Unlike conventional RF chips, the spread-spectrum waveform and currently implemented spreading code permits up to 65 of these devices to operate in close proximity without mutual interference. In difficult RF environments (e.g., multipath) the spread-spectrum waveform can typically provide a factor of 50 improvement in range and 100 in data error rates over conventional sensor units.

As illustrated in the block diagram of FIG. 13, the readings from the four analog sensor inputs 302, 304, 306, 308 (including the two external lines) are sequentially selected by the multiplexer 310 and digitized by the analog-to-digital converter (ADC) 312 subsystem. The digital data is then converted into a formatted serial bit-stream and digitally combined 318 with a user-selectable, 63-length spreading code (1 of 65 possible). The resulting direct-sequence spread-spectrum data stream [similar to those in advanced “CDMA” cell phones and in Global Positioning System (GPS) data streams] is used to phase-shift-key (PSK) an onboard synthesized 320, crystal-locked 316 916-MHz oscillator (in the right-hand “mixer” block 322) to generate the final RF transmit; a final on-chip RF power amplifier feeds 324 the antenna port 326. The user can fully control the sensor measurement rate, power consumption, output frequency, and RF spreading codes via simple connections to the chip's pads. Key MIST technical specifications are described in the incorporated publications. The only external components required for a minimal system are an antenna 330, a tiny battery 288, and an inexpensive frequency-reference crystal 284. An external receiver module interfaced to a laptop computer recovers the spread-spectrum signal and displays the sensor data; this setup was employed for the MIST system field testing to verify performance in adverse RF environments. The MIST chip is also networkable in that individual telesensors in close proximity can be programmed to operate as a multidrop wireless bus. The 3 mm ×3 mm size of the MIST telesensor micro-chip and other features of the MIST micro-chip allow it to be key component for the telesensor implants.

The ORNL MIST device is operable from roughly −25 to +70 degrees Centigrade ambient; its onboard thermometer has been verified to within 0.5 degrees Centigrade and is available in both industrial and medical versions.

A paper provided as described by ORNL to interested parties, entitled “Multichannel Integrated Spread-spectrum Telesensor (MIST) Chip,” provides further information on the MIST chip and is incorporated herein by reference.

The literature provided by ORNL also described integration of their MIST controller and spread-spectrum transmitter technology with other sensor devices, including blood oximeters and micro-cantilever based sensors. ORNL has also developed sensors capable of monitoring for presence of certain deoxyribonucleic acid (DNA) components. An additional paper provided by ORNL by Ferrell, T. L., et al, “Medical Telesensors,” referenced earlier herein describes the implementation of a pulse oximeter which has been used in conjunction with their telesensor chip technology. This paper is also incorporated herein by reference. A figure in the paper by Ferrell (his FIG. 3) shows that the pulse oximeter sensors, with appropriate circuit modifications, could also be used to monitor the pulse rate of an animal.

ORNL has filed patents for innovations they have incorporated into the MIST micro-chip and related sensor technologies, and has publicly announced the availability of the MIST micro-chip technology for licensing. ORNL patent applications further disclosing the MIST technology are incorporated herein by reference and include ERID 0538 “Short-Range Radiolocation System and Methods,” ERID 0642 “Fast Synchronizing High-Fidelity Spread Spectrum Receiver,” ERID 0656 “Wireless Spread-Spectrum Temperature Telesensor Chip with Synchronous Digital Architecture,” ERID 0678 “Hybrid Spread-Spectrum Technique for Expanding Channel Capacity,” ERID 0679 “Improved Digital Data Receiver Synchronization Method and Apparatus.”

The ORNL MIST micro-chip technology is used in a preferred embodiment of the instant invention, however, other similar electronic chips incorporating the functions described earlier, and implemented in design rules providing LSI, VLSI, or even higher levels of circuit integration, and providing very low power utilization when not transmitting, may also be used. Although the prototype wireless telesensor chip illustrated in FIG. 13 was fabricated in approximately a 3 mm by 3 mm format, this layout has not been optimized to achieve minimum size, and the layout illustrated also contains several test pads used to support prototype development and testing which will not be needed in a production version of the telesensor micro-chips. The layout of the micro-circuits and/or components used on the micro-chip can be altered to provide a different format. For example, in order to provide a micro-chip more compatible with implant injection by a needed, the layout of the chip may be altered into a more slender format, for example 2 mm by 4 mm, or 1.5 mm by 5 or 6 mm.

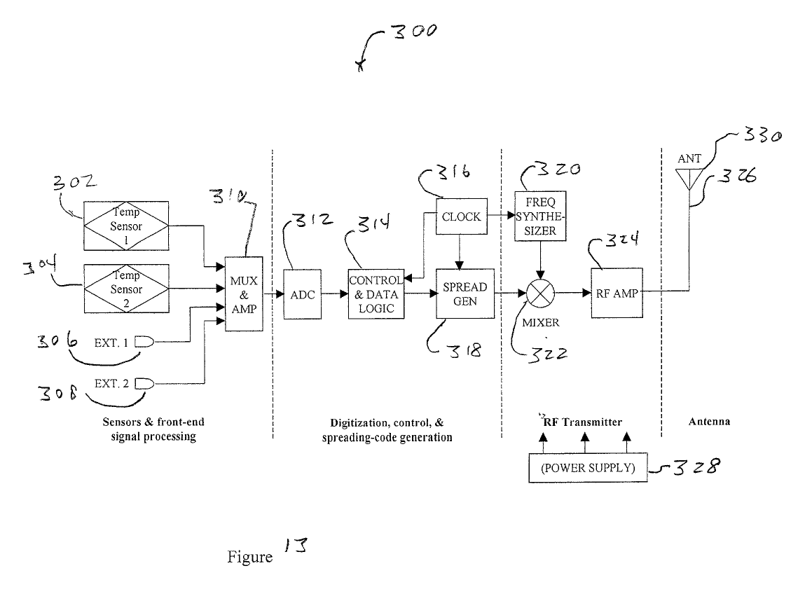

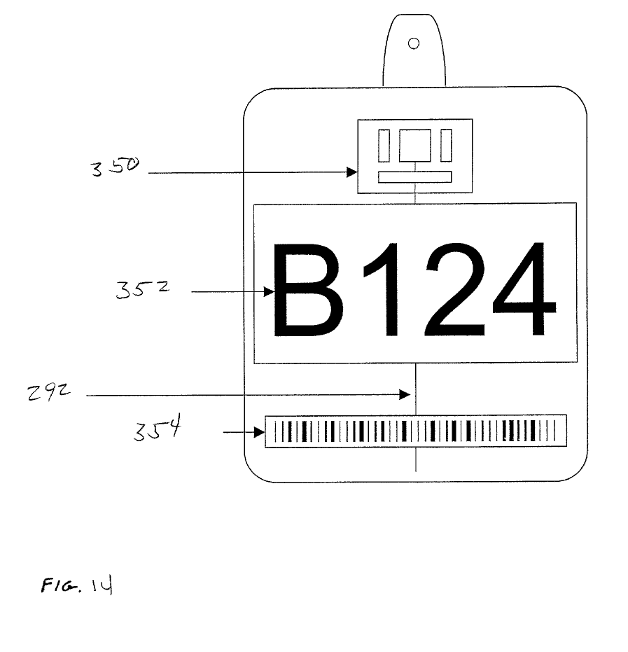

The electronic components described above, or simpler electronics in some cases, can be used to implement dumb or smart wireless implants. These or similar electronics can also be used to implement different telesensor configurations such as a configuration employing the telesensor electronics described above integrated into an ear tag similar to that illustrated in FIG. 14 and 15 and attached by wire to a temperature or blood oxygen sensor installed within the ear canal of the animal being monitored. Electronic microchips such as those described above, along with related components also noted earlier, may also be mounted on ear tags, collars, or otherwise attached external to the animal being monitored and used to receive and relay signals from other telesensor implants installed within the animals.

Dumb wireless telesensor implants, in our terminology, are telesensor implants which simply power-up sensor circuits at preprogrammed intervals, make a measurement, then power-up transmitter electronics and transmit the results of the measurements to a receiver and processing unit which monitors for an abnormal condition or simply records and stores measurement results for later use. The dumb wireless telesensor implants can employ spread spectrum waveforms or more conventional data modulation and transmission techniques. Dumb wireless telesensor implants which have a receiver can also receive and execute simple commands, including execution of a measurement cycle and transmission of the measurement results.

Smart wireless telesensor implants wake up at designated times, make measurements of one or more parameters, store and analyze the new and previous data to determine if an abnormal condition exists. Smart telesensor generally power up the transmitter circuitry and transmit when an abnormal condition exists or when they have been otherwise programmed to transmit measurement and/or analysis results. Smart telesensors can also alter their measurement intervals and transmissions based upon results of measurements. Smart telesensor embodiments which have a receiver can also respond to commands issued by an external source, or they may receive data from external sources on the ambient environment parameters and employ such data in determining when an abnormal condition exists which warrants transmission of an alert warning.

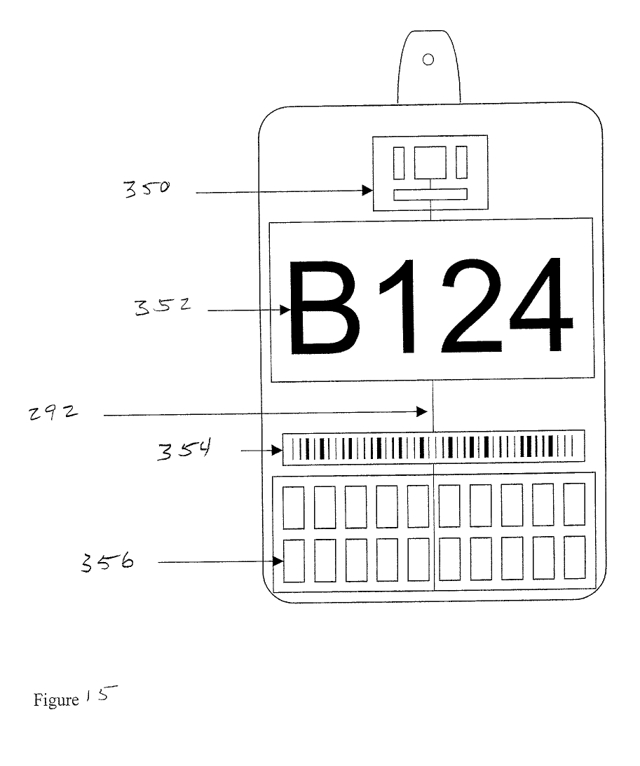

As noted earlier, in addition to a wireless telesensor micro-chip based on the ORNL MIST technology, other components included in the electrical and electronic portions of a typical telesensor implant of the instant invention, as illustrated in FIGS. 16 through 19, include one or more batteries 288, a reference oscillator 284, an EPROM chip or other memory storage chip or device 286, an antenna 292, a circuit board or other interconnect apparatus 294 and miscellaneous coils 287 and capacitors 289.

Telesensor Implant Interfaces with Animal Bodies

Different implant designs may be employed to accommodate implant placement in different locations on different animals. FIGS. 12, 17 through 22 illustrate examples of implant configurations which may be injected or otherwise installed entirely within the animal (i.e., under their skin or within tissue or bone), placed within the ear canal or other cavity of the animal accessible without penetrating the skin of the animal, including being swallowed as a pill or capsule, or installed percutaneously (i.e., being installed such that a portion of the telesensor implant is installed within tissue or bone or otherwise below the skin of the animal, and a portion of the telesensor implant, for example, an antenna and, in some embodiments, additional wiring, penetrates the skin of the animal and is exposed outside the skin of the animal). A percutaneous implant presents enhanced risks of infection and requires additional features and measures, as described later herein, to help reduce such risks. However, a percutaneous implant which enables use of an external, exposed antenna may offer significant offsetting advantages in terms of increased transmit and reception range and other benefits.

In animal product production environments and in animal care environments where products from the animal being monitored (e.g., milk, antibodies), or all or portions of said animals, may ultimately be processed into food for humans or other animals, components and materials used within the telesensor implants should be non-hazardous (i.e., non-toxic, non-carcinogenic, and not producing other harmful effects) to humans and animals, or special measures must be implemented to insure the animals being monitored are protected from exposure to hazardous materials during use of telesensor implants, and that any hazardous telesensor implant materials are recovered before or during slaughter or processing of food products from the animal. Although some hazardous materials, such as metals and some electrolytes used in some battery technologies (e.g., lithium, cadmium), and including but not limited to other potentially toxic, carcinogenic, or otherwise harmful materials, can be suitable encapsulated to prevent risk to the animal in which they are installed, it is desirable in implementing an AAHMS system, particularly in an animal product production environment, to consider (1) the cost of recovering the implant before or during slaughter and processing of the animal, (2) the risk for product contamination if the implant is not recovered, or (3) the use of configurations, materials, and implant locations where recovery or removal of telesensor implants may not be necessary.

The need to insure food products are not contaminated by unacceptable levels of chemicals, or by objects or substances which could cause injury or other unacceptable response in human or animal consumers of the food products, imposes additional considerations in implementing some embodiments of the instant invention. Although an implant may be injected into a portion of an animal which is not used for human food, past experience with electronic implants used for animal identification and other purposes has shown that, for at least some implant designs and implant locations, the implants can drift from the original implant location to other portions of the animal's body. In addition to the likelihood that parameter measurements made by a telesensor implant may be misleading if the implant migrates from its original implanted location, there is another possibility that, although an implant is installed in a portion of an animal not processed for human food, the implant will end up in a location which may be processed into a food product for human or animals. It is also common practice in processing beef cattle that those portions of the animal which are not processed into cuts of meat (i.e, steaks, roasts) or other products (e.g., ground beef, hot dogs) for humans are processed into food for other animals. This includes portions of the meat and other organs which go, for example, into pet food, as well as bone, connective tissue, and other by-products which are ground up into a meat and bone meal product which may, in some cases, be fed to other animals (e.g., chickens, swine).

While it is generally unacceptable for any portion of a foreign object to end up in a food product destined for humans and most pets, it may be acceptable in some cases for telesensor implants to be ground up along with the other animal processing by-products into a meat and bone meal product or similar by product from animal product processing, so long as the materials are not toxic or otherwise harmful. This may be permitted on a routine or an exception basis. The exception basis being where procedures exist which recover most of the implants from the product stream, but, due to quality control, implant migration, and other reasons, some implants slip through the recovery or removal process and end up in the final product. Thus, some embodiments of the instant invention are disclosed herein which can be implemented with low-toxicity materials, or with low amounts of such materials, and with other features which provide a low risk of migration of the implant within the animal being monitored. These features may provide greater flexibility and acceptance by regulatory agencies and consumers for use of the telesensor implants in some animals used for food or other sensitive products for humans or other animals.

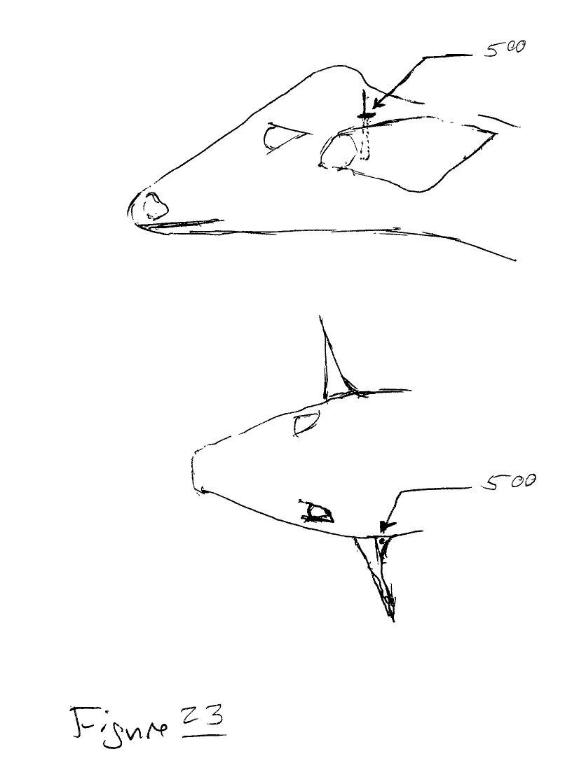

In cattle (e.g., both beef and dairy), one preferred location 500 for injection of telesensor implants, which provides a reasonable balance among multiple requirements, including the need to provide a reasonable measure of animal core temperature as well as being preferably located in a portion of the animal not normally processed for human food, is the location just behind the ear attachment of the animal, close to the skull as illustrated in FIG. 23. This area is accessable, for example, from above and behind the ear and contains primarily cartilagineous material and muscle, but with sufficient blood vessels to provide a reasonable measure of temperature, blood oxygen, and some other parameters. Other locations on an animal may also be used for implant locations in addition to or in place of the area behind the ear. As noted elsewhere herein, for some applications and embodiments, the electrical and electronic components of the animal may also be suitably encapsulated and incorporated into a capsule which can be swallowed or forced into the intestinal tract of the animal where it can provide a measure of animal core temperature and, in some embodiments, other parameters, as it passes through the animal.

There are other animal production and animal care environments where the animals being monitored by an AAHMS implementation are not processed for food products for humans or other animals. In such applications, the use of non-hazardous materials is less of an issue so long as telesensor implant materials used within the animals being monitored can be reliably encapsulated by suitable encapsulant materials to preclude leakage into the animal's body or other contact of animal tissue or fluids with the hazardous materials.

The principal typical components of a telesensor implant affected by concerns for toxicity and other contamination are the battery components and some candidate encapsulant materials, such as glass. These components could present a hazard to human consumers and other animals from biting down on intact or broken portions of telesensor implants, or from chemicals released if a telesensor implant including the battery is ground up during processing.

Some of these concerns can be reduced, in some embodiments, by use of appropriate encapsulants and by use of low-toxicity battery technology, such as is available with some types of plastic batteries. For embodiments where low toxicity is desirable, it will still be necessary to also pay attention to the selection of other materials used within telesensor implants (e.g., circuit board traces, materials used within the electronic chips and mounts, adhesives), but reduction of battery toxicity and risks from materials such as glass encapsulants will offer significant benefits.

The outer layer or layers of material 296, 360 surrounding the telesensor implant, which may be exposed to animal tissue or fluids, should be biocompatible, and in many cases, should promote the ingrowth of tissue to help anchor the telesensor implant in the desired implant location. For percutaneous implants, the outer layers of material surrounding the telesensor implant should also promote healing and sealing at the point of skin or hide penetration by promoting tissue ingrowth and adherence to the outer layers of the implant materials. These biocompatibility objective stated above may be met by use of such materials as medical urethane compounds, parylene, or other biocompatible materials. The tissue ingrowth and anchoring objectives (and sealing objective for percutaneous implants) may be met by use of collagen sponge materials and other materials arising from tissue engineering and burn healing technologies such as disclosed in U.S. Pat. No. 5,629,191, U.S. Pat. No. 5,833,665, and U.S. Pat. No. 5,997,895 (see References), or by use of medical polyurethane foams or other materials such as used for long term installations of catheters or wiring in dialysis patients and patients with artificial hearts. Collagen sponge materials and related materials which may promote tissue ingrowth and prevent infection are available, for example, from Integra LifeSciences, Plainsboro, N.J., and Thermedics, Inc. Integra LifeSciences markets a device called VITACUFF (trademark) which incorporates such materials into a device used in long term emplacement of catheters through the skin of humans. Medical polyurethane materials and similar materials are available from Thermedics, Inc., and from other organizations supplying such products to the medical research and health care communities. In some embodiments, disinfectants or antibodies may be incorporated into special coatings included on the telesensor implants in order to help control pathogens which may be introduced during installation of the telesensor implant.

Alternative Telesensor Implant Configurations

Wireless telesensor implants can be fabricated in different configurations for different applications and embodiments. Telesensor implants generally comprise the electronics and electrical components as described above together with other materials which provide for the interface with the animal. As noted earlier, telesensor implants can be configured into different shapes and made of different materials, or combinations of materials, as needed to provide use in different locations in different sized and types of animals, and to provide optional embodiments which can be tailored to different needs of different animal production and other animal care environments.

By way of examples, FIGS. 12 and 17 through 22 illustrate alternate embodiments of telesensor implants which may be used for injection into an animal, insertion into the ear canal or other cavity of an animal, or for a percutaneous implant (i.e., with a portion of the implant internal to an animal and a portion of the implant penetrating the skin or hide of the animal and exposed on the outside of the animal). The configurations illustrated are not mutually exclusive. Many of the features of the implants illustrated in these figures can be mixed and matched in different combinations to achieve different purposes in different applications and embodiments of the instant invention. Some embodiments may employ high density energy storage battery technologies such a nickel metal hydride or lithium ion 288, plastic battery technologies, or more conventional battery technologies 358. Electromagnetic pickup coils or other energy collection devices, including the normal signal transmission and reception capability, may be incorporated along with appropriate circuitry to provide a capability to recharge batteries in the device either before installation into an animal, while installed in an animal, or after removal from the animal. Coils may also be used in some embodiments to provide an alternative means of triggering a readout to reduce drain on batteries or to provide a risidual readout capability in case of battery failure. In some applications and embodiments, piezo-electric microcantilevers and similar devices capable of extracting energy from the movement of the animal may also be used along with appropriate circuitry to provide a battery recharge capability.

Representative Injectable Implants

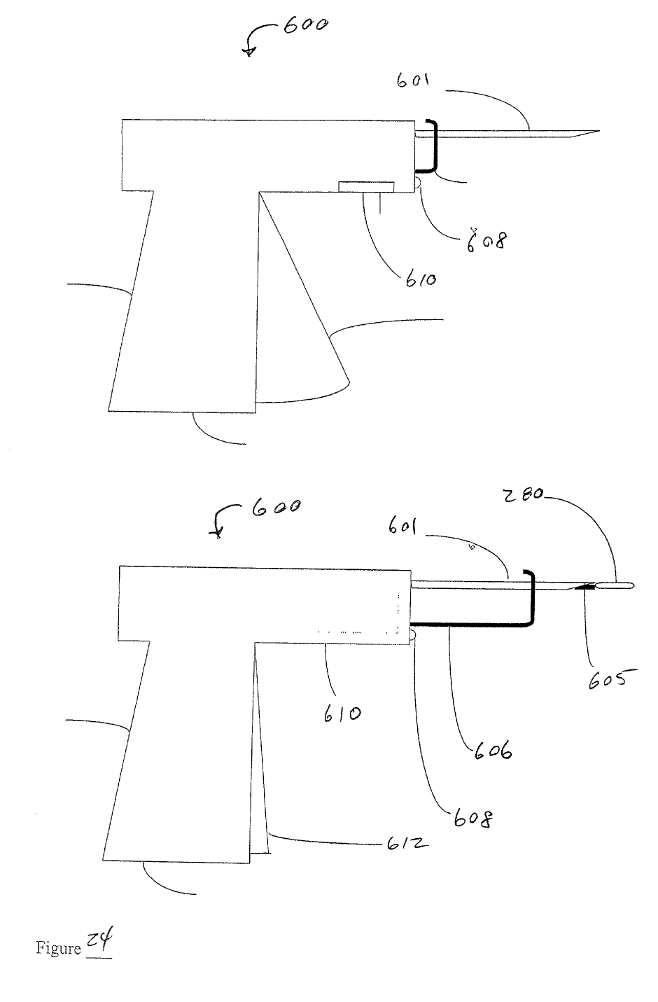

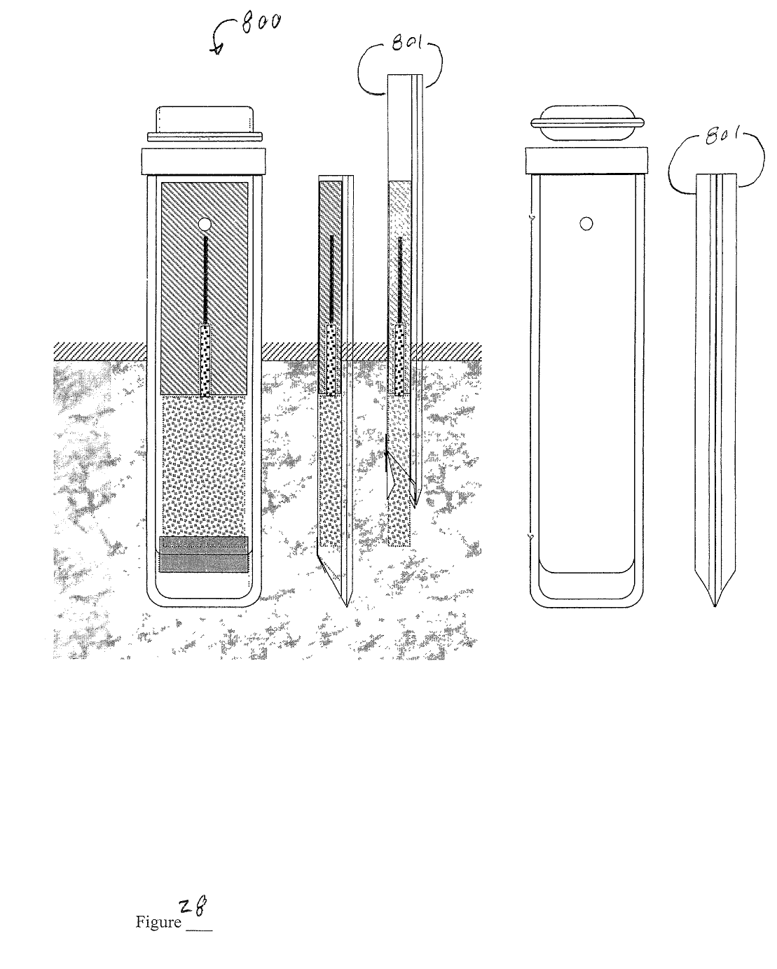

FIG. 12 illustrates one embodiment of a telesensor implant which is tailored for injection into an animal to be monitored using a needle. It this particular case, the implant is more or less cylindrical and may typically be about 2 mm in diameter and 6 to 10 mm long. It would be injected by an installation tool similar to that illustrated in FIG. 24. Such an implant would typically come pre-packed in the needle which will be used for injecting the implant, and the needle would be designed for quick attachment and detachment from the installation tool. The implants may also be packaged in a form which promotes efficient loading into a needle at the installation facility just before injection, as illustrated in FIG. 28.

The electrical and electronic components of the telesensor implant may be encapsulated in glass or another biocompatible material to protect the electronics from body fluids and tissues, and to protect body fluids and tissues from any potentially hazardous contamination from materials in the implant.

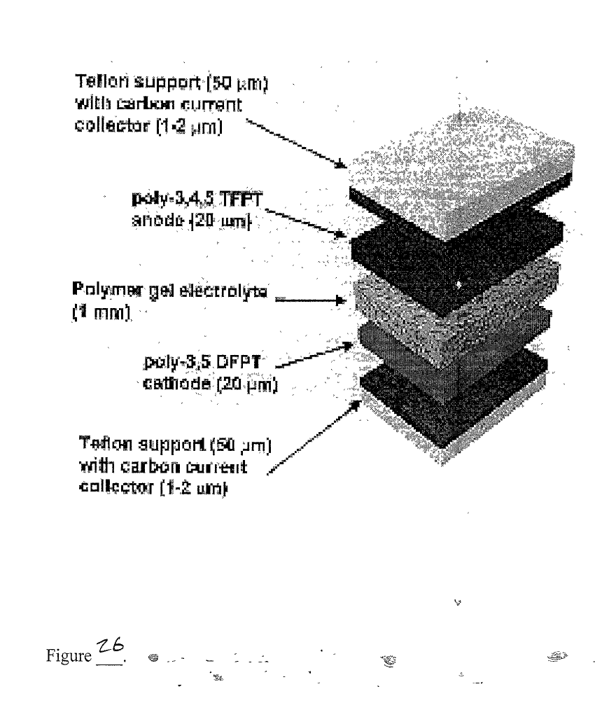

Plastic battery technologies recently developed by The Johns Hopkins University (see References in Appendix B and FIG. 26) and others offer advantages for some embodiments of telesensor implants. FIG. 26 illustrates a typical layered approach for plastic battery technology. In a typical configuration, a plastic battery can be implemented in a flat sheet approximately one millimeter thick as illustrated in FIG. 26. In other configurations, the dimensions of the layers and overall thickness of the sheet battery can be modified to suit needs of a particular application. Plastic battery sheets can also be rolled into generally cylindrical forms, or can be molded in many other shapes. In some formulations, plastic battery technologies offer good energy densities implemented with low toxicity compounds. For some applications and embodiments of the present AAHMS invention, the lower toxicity and other attributes of telesensor implants using plastic battery technologies may reduce the need to recover the implants from animals or from some of the product streams (e.g., meat and bone meal by-product) resulting from processing of animal products. Plastic batteries can be formulated for one-time use or as rechargeable batteries.

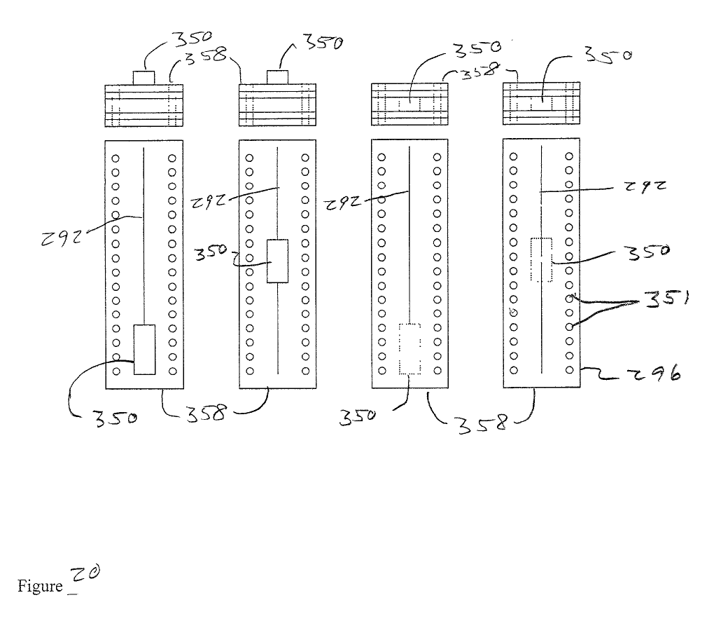

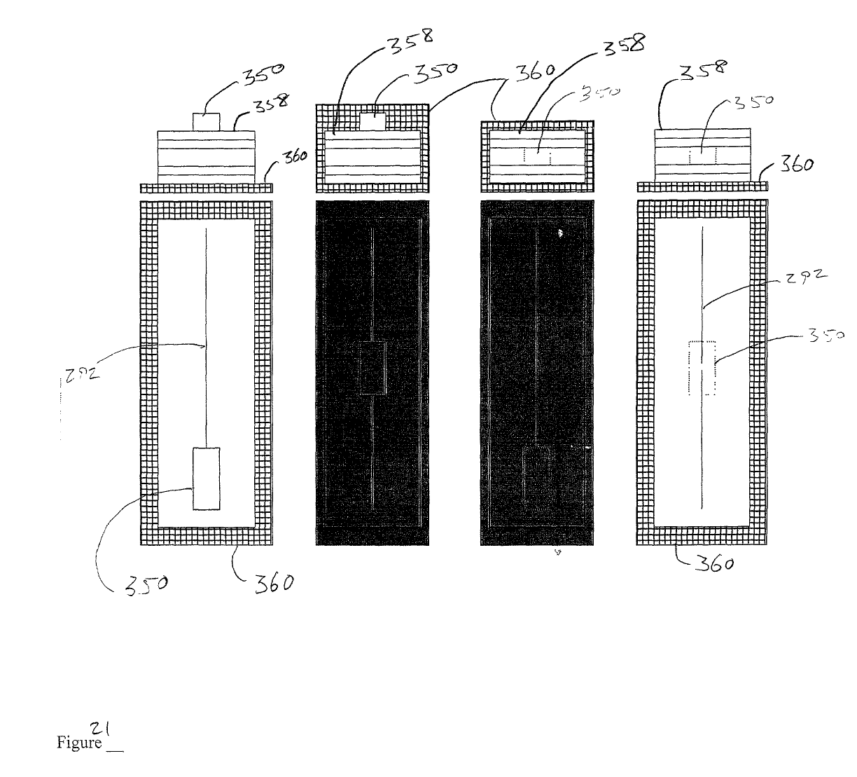

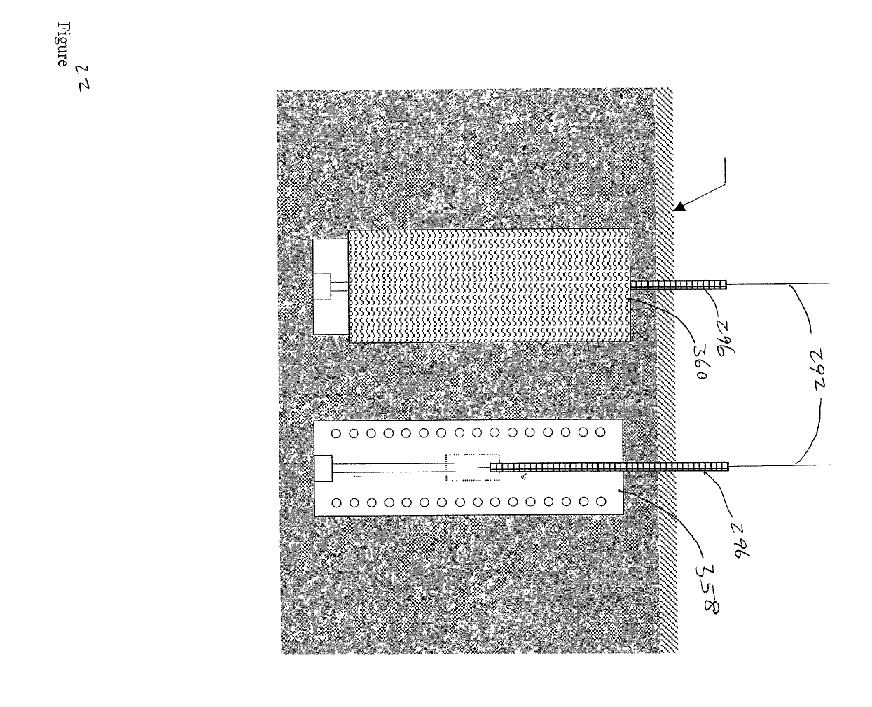

Examples of telesensor implants implemented with plastic battery technologies are illustrated in FIGS. 20, 21, and 22. In these implementations, as examples, the other electrical and electronic components 350 of the telesensor implants may be attached to one side of or sandwiched between layers of plastic battery materials 358, or cavities for the other electrical and electronic components may be molded into the battery layers or material to achieve a desired external form factor. The plastic batteries may be formulated with an outer envelop 296 which is biocompatible, or enclosed in other layers of materials 360 such as surgical mesh which provide desired biocompatibility and other properties. In some embodiments, such telesensor implants may be enclosed partially or completely with polyurethane foam, collagen sponge, surgical mesh, or other materials which may promote tissue ingrowth or attachment of tissue to the implant which helps anchor the implant into a desired location within the body and prevent migration of the implant. In other embodiments, holes 351 may be formulated into the plastic battery materials, as illustrated in FIGS. 20 and 22, or surgical mesh or other materials 360 may simply be attached to one or more sides of the plastic battery as illustrated in FIG. 21 to promote tissue ingrowth or attachment.

Typical dimensions of the telesensor implants formulated with plastic battery technologies may be on the order of one-quarter to one-half inch wide, less than one-quarter inch thick, and one or two inches long. Larger or small configurations may also be employed in different embodiments of the instant invention to accommodate tradeoffs in battery life, antenna size, and the size of implant which can be accommodated in different animals and different implant locations on animals.

Additional coatings such as a polyurethane foam or collagen sponge may be added to the telesensor implants in some embodiments to promote the attachment of tissue to the implant coating, or the ingrowth of tissue into the coating material in order to help anchor the implant within the body of the animal and reduce migration from the intended location.

For implants containing IR or other optical communications transmitters or receivers, or implants containing IR or optical sensors (e.g., blood oximeter as disclosed in the attachments, which may also be used as a pulse rate sensor), it may be necessary to either select encapsulant and coating materials which are transmissive at the wavelengths of interest, or provide windows in the encapsulant materials and any other special coatings which windows are transmissive at the wavelengths of interest. Alternatively, optical fibers can be integrated into the implant construction to provide an optical pathway from an IR sensor through encapsulating material to surrounding tissue, or from an optical/IR transceiver through encapsulating material, and through the hide or skin of the animal, to the external environment.

In embodiments employing micro-cantilevers and other miniature devices as vibration sensors and/or accelerometers, it may be desirable to employ coatings which also have an appropriate acoustic impedance and related properties to help insure small vibrations are transmitted from tissues to the sensing elements of the telesensor implant.

The battery used in such an implant, or other telesensor implants, may employ its own independent encapsulation if warranted by its material and construction requirements, or the encapsulation of the battery may be integrated with the encapsulation provided to other portions of the implant.

In some embodiments, it may be desirable to employ metallic components as portions of the encapsulating materials, but having provisions for radiation and reception of RF or optical/ IRelectromagnetic energy into the telesensor implant for communications, sensing, and or battery recharging purposes. In one example, the telesensor implant may include a metallic envelope divided into two capsules, somewhat similar in shape to the two ends of a gelatin capsule such as is commonly used in construction of pills for delivery of medicines, but with the two metal ends joined in the center or elsewhere by a dielectric sleve or other dielectric separation so that the metallic ends may be used as an electromagnetic antenna for sensing, communications, or other purposes.

Implants for Ear Canals or Other Body Cavities

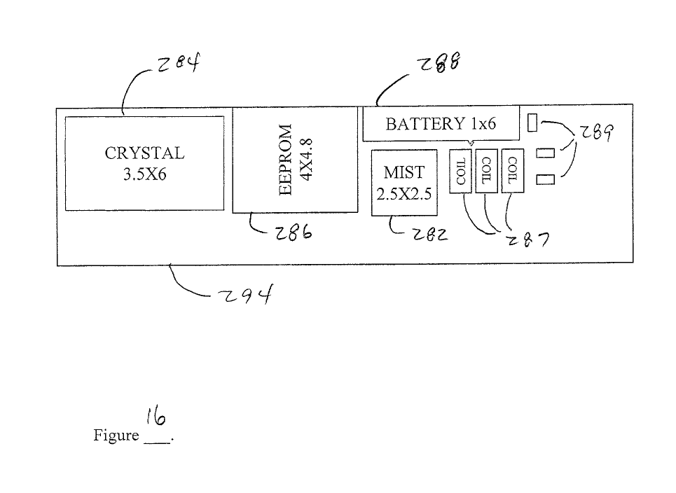

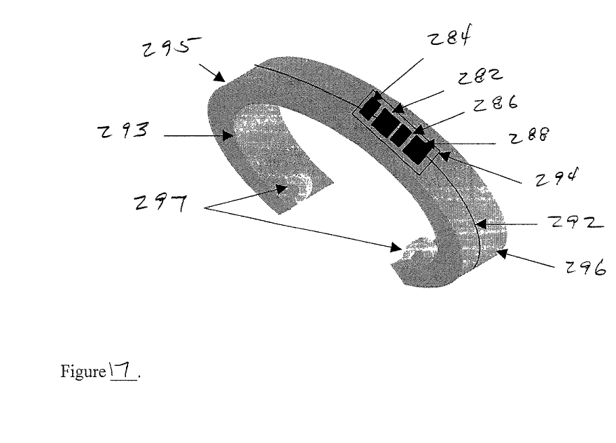

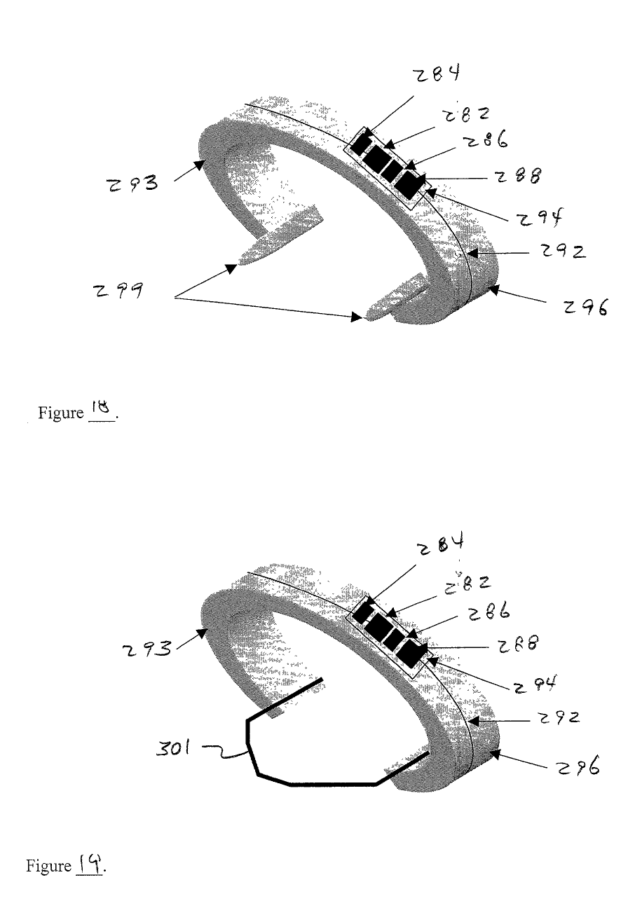

FIGS. 17, 18, and 19 illustrate, as examples, configurations of telesensor implants which can be inserted into an ear canal or, with appropriate modifications, another cavity (e.g., rectum, vagina, nasal) of animals to be monitored. For such applications and embodiments, the telesensor implant may contain a curved member 293 of plastic or other material. The electronics 282, 284, 286, 288 and other electrical components 294, 292 may be affixed to the outer diameter surface of the curved member 293, the inner diameter, embedded within the material, or otherwise distributed on or within the curved member material. The curved member may be made of a plastic having a springlike action so that the curved member can be compressed into a smaller diameter to support installation, and then expand to a larger diameter after emplacement into the ear canal or other cavity, similar to the action of a snap ring. Alternatively, the curved member 293 may be made of a resilient, compressible material, such as a foam rubber, sponge or other material which can be compressed to support installation, then expand into place to support retention within the ear canal or other body cavity. In some applications, the outer surface of the implant may be coated with an adhesive to aid in retention of the implant within the animal. Such implants may contain other features which aid in the installation or retraction of the telesensor implant. For example, loops 297 may be added on the inner diameter of the curved member as illustrated in FIG. 16 to permit installation or removal of the implant using a tool similar to snap ring pliers, suitably modified to reduce risks of puncturing an eardrum or causing other damage to the animal being monitored. FIG. 18 illustrates the addition of tabs 299 to the sides of the curved member to permit grasping and removal of the telesensor implant for removal purposes. FIG. 19 illustrates the incorporation of a wire member 301 with the curved member to provide another means by which the telesensor implant could be grasped by pliers or a tool containing a hook to support removal of the implant. The wire could also be shaped so that it could be inserted into a tube, thereby compressing the outer diameter of the telesensor implant to support installation. During installation, a pushrod within said tube could push the wire out, thereby allowing the telesensor implant to expand into place. In some embodiments, a wire member, similar to that illustrated in FIG. 19, used to support installation or extraction of the implant, could also be used as part of the antenna system on the telesensor implant. Special coatings may be added to the implant in some embodiments to promote long term compatibility with tissues in the ear canal or other cavity without causing necrosis or other damage or irritation to such tissues. For some types of sensors, materials may be selected, or holes or windows provided in the covering of the implant, to permit transmisison of infrared or other signals needed to support implementation of some sensor functions. For embodiments of the instant invention where it is desirable to monitor the temperature of the animal being monitored, a preferred location of the installed implant is deep within the ear canal adjacent to the ear drum. This location provides a relatively good measurement of the core temperature of many animals. Such implants may incorporate thermistors, thermocouples, or other contact type temperature sensors, or such implants may also incorporate infrared sensors, including infrared sensors based upon micro-cantilever and atomic force microscope technologies as developed by ORNL and others (see References). In some embodiments, the curved member 293 noted above may be made wholly or partially of plastic battery material to provide energy storage in place of, or in addition to, other battery forms as illustrated in FIGS. 17 through 19.

Percutaneous Implants

Percutaneous implants are illustrated, as examples, in FIG. 22, and their installation is illustrated in FIG. 28, and discussed further above. One key advantage of a percutaneous implant is that an antenna may be exposed on the outside of an animal, thereby enabling use of a longer antenna than it may desirable to implant inside an animal, and also avoiding the attenuation and distortion of RF signals by the tissue of the animal which is a problem at some frequencies. In some embodiments of a percutaneous implant, solar cells in the form of a flexible flat disk or other shape may be added on the exposed portion of the animal to permit use of solar energy to charge or otherwise power the telesensor implant.

Programming, Calibration, Recharging, and/or Activation Units

Some applications and embodiments of the instant invention will have a unit separate from the installation tool to support customized programming of the telesensors before installation, to support calibration of telesensors when a particular application demands enhanced precision, to support charging or recharging of batteries contained in the telesensor, and/or to support activation of telesensor units which may be shipped with batteries disconnected or otherwise disabled or isolated from telesensor circuitry to prevent battery discharge during shipping and storage.

Installation Tools

The installation tools 600, 700, 800 of the present invention provide for efficient installation of the implants of the instant invention into the animals to be monitored with minimum pain and suffering and, in some embodiments, provide efficient functional capabilities (e.g., bar code readers 608 or readout units for other electronic ID devices) for maintaining a correlation between the unique electronic identification codes transmitted by the telesensors and human readable identification codes 352 displayed on ear tags, collars, or other devices attached to the animals. In some embodiments, a bar code reader or other reader for electronic ID units may be added to the outside of tools already used for injection of electronic implants, hormone implants, or similar tools, or the capabilities to read such units may be integrated into the overall design of the tool used to support installation of the telesensor implants.

The installation tools, in some embodiments, may be designed to work together with special packaging 60 of the telesensor implants, as delivered to the animal production or other animal care operation, to provide for efficient and sterile installation of the telesensors with minimal risks of transmitting infection between animals. In some embodiments, the installation tools also provide other functions to assist in installation of the telesensor implants and minimize risks or severity of infection associated with installation of the implants. These other functions may include a capability to spray a disinfectant on the location where the implant is to be injected or inserted, and/or a capability, in some embodiments, in conjunction with the needles, blades, tubes or other elements of implant packaging, to direct a stream of air, water, or other fluid onto the area where an injection or incision supporting insertion of an implant is about to be made in order to spread the animals hair, fur, feathers or other covering away from the injection or incision site and remove foreign materials (e.g., mud, manure) so as to minimize the transmission of surface contaminants into the animal during installation of the telesensor implant. Embodiments providing a capability of employing a fluid stream for purposes of disinfection, cleaning, or diversion of hair, fur, or other covering also employ a diversion cutout or other safety feature which is activated as part of the installation sequence to insure that the aforementioned fluid stream is not directed into the animal. Allowing such a stream to be injected into the animal could cause an embolism or other serious injury or death to the animal.

Telesensor implants such as those illustrated in FIGS. 20, 21, or 22, or telesensor implants configured into other shapes as well, may be surgically implanted or may be delivered to the installation facility prepackaged into needles, tubes, or paired blades such as those illustrated in FIG. 28. The paired blades 801 illustrated as an example in FIG. 28 are sharpened on at least one end, and possibly along the sides, and may be designed in some embodiments to fit into an installation tool which vibrates or otherwise moves the blades from side to side, relative to one another, at the end of the blade so as to create a slicing action to accommodate the making of an incision and insertion of the paired blades into the desired implant location in the animal's body. Such telesensor implants may be installed internally, subdermally, or as percutaneous implants, wherein a portion of the implant, normally only the antenna, penetrates the hide or skin of the animal and remains exposed on the outside of the animal. Depending upon the thickness of the implant and other considerations, the blade pair (as illustrated on the right side portion of FIG. 28) and installation tool may be designed to permit the blades to simply separate, after the incision is made, as a pushrod pushes the implant out from between the blades as the blades are extracted from the animal. Alternatively, one end of the blades may be designed with a hinged portion (as illustrated on the left side of FIG. 28), using metallic tape or other material to provide the hinge action, which opens to permit the telesensor implant to be pushed out by a pushrod and remain in the animal's body, internally or percutaneously, as the blades are extracted. Depending upon the fragility of the collagen sponge, foam, or other material optionally used as a covering on the implant, an additional pushrod extension may be included in the blades or tool which slides along beside the telesensor implant to open the hinged portion of the end of the blades to prevent damage to the telesensor implant as it is pushed from the tool so as to remain deposited in the animal during extraction of the blades.

In some embodiments employing relatively fragile coating foams and sponges, it may be necessary to employ special features and techniques in the installation needles and other tools to insure the coating material is not damaged. These features and techniques may include using an injection technique wherein the needle containing the implant is injected into the tissue of the animal, then a pushrod in the installation tool pushes the implant from the needle so that it remains in the tissue as the needle is retracted from the animal.

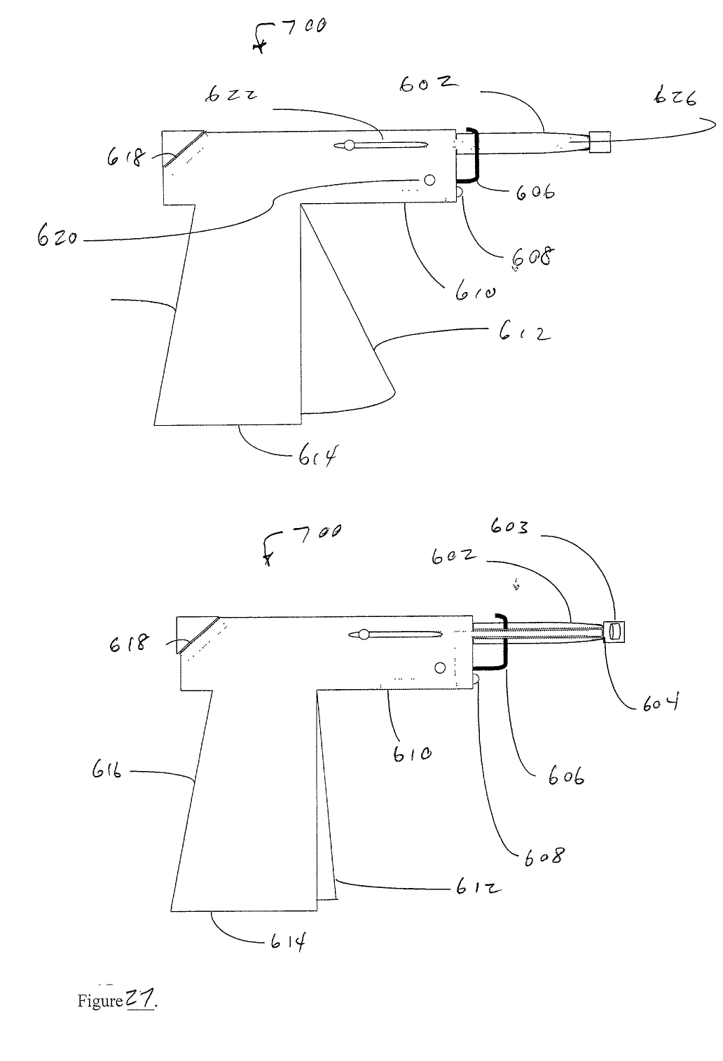

In some embodiments of the instant invention, a special tool, such as that shown for example in FIG. 27, may be used to support installation of the curved implants into an ear canal or other body cavity. In a preferred embodiment, the curved implants may be delivered to the installation site or facility in special packaging wherein the telesensor implants are already pre-compressed for easy loading into a tube or other member of an installation tool.

Where long term storage of implants in a compressed form may cause them to take a set or otherwise impact the resiliency and ability of the implant material to expand back to a larger outer diameter, the packaging may be designed to support compression of the impants to a smaller diameter as they are loaded into the tube or other member of the installation tool. In some embodiments, the implants may be delivered in special packaging wherein the implants are already pre-loaded each into its own tube of metal, plastic, or other suitable material, which tubes are themselves designed for easy attachment to and removal from a separate installation tool, similar to that shown, for example, in FIG. 27.

In some embodiments, a miniature camera 626 similar to that used in fiberscopes, laproscopic surgery, and other applications may be incorporated into the installation tool and tube, preferably into the center portion of the tube, so as to provide imagery to the tool operator to support installation of the implant into the desired location with reduced risk of injury to the animal. The display 618 may be incorporated into the end of the tool, using, for example, a display technology such as active matrix displays used in palm computers and virtual reality headsets, or the display from said miniature camera may also be presented on a video monitor or other suitable display device easily observed by the installation tool operator. For some installation applications, the installation tool may incorporate an adjustable depth gage 606 or other device to help prevent the installation tube from being inserted below a certain depth in the animal to reduce risks of injury to the animal, particularly if the head or other portions of the animal are not well restrained during installation.

During installation of the telesensor implant into an ear canal, the tube containing a compressed telesensor implant, such as those illustrated in FIGS. 17, 18, and 19, is inserted into the ear canal or other cavity to an appropriate depth as determined by experience and observation of the operator, depth gages, and/or imagery provided by a miniature camera. A trigger 612 or other lever device on the tool is then activated or operated by the tool operator to cause a pushrod 605 or other mechanisms within the tool 700 and/or tube to push the telesensor implant out of the tube and into the ear canal or other cavity. Upon ejection from the installation tube 602, the telesensor sensor implant then expands so as to become fixed within the cavity, or a special adhesive (e.g., an encapsulated cyanoacryllic adhesive) on the outer surface of the implant bonds with surrounding tissues to fix and retain the implant into position.

Identification and/or Relay Tags

For many applications of the instant invention, a method is required to insure that, in a crowded animal production or other animal care environment, when a telesensor of the instant invention transmits data or transmits a warning alert indicating that the animal in which, or on which, it is installed requires attention, positive identification of the animal can easily be made by a human attendant. In preferred embodiments for many applications, a tag or label (1)containing a human readable identification code 352 (e.g., human readable alphanumeric symbols, other symbols in any language, or color codes) and (2)which can be affixed to the animal in some manner (e.g., ear tag; tag or label on collar, bracelet, harness, anklet, horns, skin, or hide) is used for that purpose. In some embodiments, the tag or label may contain a bar code 354, RF tag, identification chip, or other device containing a machine readable identification code to support efficient scanning and correlation of the human readable identification code on the tag or label with the identification code(s) programmed into any telesensor(s) installed on the animal. For some animals, it may be necessary or desirable instead of or in addition to use of tags, collars, and other human readable ID devices noted above, to use or apply an identification code comprising a tattoo, stencil, colored dye pattern, or other such means of identification.

Communications Relay

In some embodiments, the same tag or label as used for visual identification purposes, and thus attached to the animal, (e.g., an ear tag on livestock) may also contain solar cells 356 and/or one or more batteries and micro-chips and other components 350 which provide a communications relay function. The communications relay function may provide a simple boosting of the signal transmitted by an implanted telesensor (e.g., a simple transponder), or the communications relay function may provide for detection and processing of data transmitted by an implanted telesensor. Such processing may, for example, employ measurement data from sensors integrated into the tag or label (e.g., sensors to measure ambient environment parameters such as temperature, humidity, solar radiance) together with data from a wireless telesensor, or other sensors attached by wires or optical fibers, to determine whether a warning alert or data should be transmitted directly to an animal attendant or to a central control and monitoring station.

In some embodiments, as indicated earlier and in FIG. 3, telesensor microchips and other components installed on an external ear tag 51, collar, adhesive skin patch, or other externally mounted device may be connected by wire to a sensor located in a cavity such as an ear canal, vagina, rectum, or other location as necessary to measure one or more parameters related to the health and status of the animal. The telesensor micro-chips installed on the external tag, collar, patch, or other external attachment device monitor signals from the sensors connected by wire and provide the sensing, processing, transmission, and/or reception functions as described elsewhere herein for other telesensor unit configurations.

Special Packaging

In some embodiments, to enhance efficiency and sanitation in installing the telesensor units (implants or external units) into or upon the animals to be monitored, while also providing appropriate means for correlation of human readable identification codes with identification codes contained in the telesensor units, special packaging 60 of the telesensor units may be employed. Such special packaging may be tailored to maintain sterility and support efficient, automated installation of the telesensors in the animals and to support identification code tracking of the implant in animal care environments involving large numbers of animals. In some embodiments, the special packaging may consist simply of including with each telesensor implant a tag or label which can be affixed to an aforementioned external attachment device on the animal at the time of telesensor installation. In such cases, the tag or label may contain the same identification code as programmed into the telesensor, or a different code which has already been correlated through an entry in a database or other data set with the identification code programmed into the telesensor.

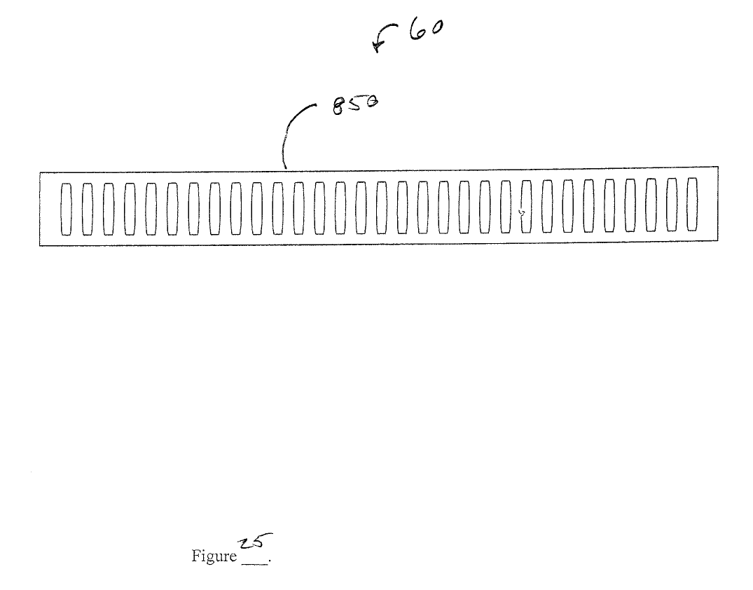

The telesensor implants may be packaged in protective packaging 850 as illustrated in FIG. 25, which would permit individual implants to be easily loaded into the needle, tube, or other receptacle of the installation tool. Alternatively, to avoid possible transmission of diseases between animals by use of the same needle, tube, or other delivery device, the packaged implants may be preloaded into needles, blades, tubes, or other delivery devices which can be readily attached to an installation tool, for example a tool similar to those illustrated in FIGS. 24 and 27 via a quick attachment or disconnect mechanism (e.g., compound threads, bayonet mount, miniature air-hose ball and socket type fitting).

The special packaging may also include other features to promote efficient installation of the telesensor units with minimal risk of infection to the animal. For example, telesensor implants may be packaged within the needle, tube, paired blades or other device which may be used to help install the telesensor implant into the animal. The needle, tube, paired blades, or other device may be designed to provide a self-contained installation capability for the implant, or they may be designed for quick attachment and disconnect from an installation tool designed to promote efficient installation of the telesensor implants. The needle, tube, paired blades, or other device may be designed to be disposable or may be designed for recycling and reuse. In some embodiments, the design and construction of the needles, tubes, paired blades, or other devices may provide a capability for the use of fluid streams for application of disinfectants, cleaning, or diversion of hair, fur, feathers, or other potential sources of contaminants from the site of the injection or incision. Such a capability may employ, for such fluids, special passageways provided within the needles, tubes, paired blades, or other devices used for injection or insertion of telesensor implants into animals, or into cavities of animals.

The special packaging may also contain a bar code or other machine readable identification code affixed on or near each telesensor to permit scanning and correlation with the human readable identification code affixed to the animal.

Wireless Receiver, Transmitter, Transceiver, and/or Transponder Units

Some embodiments of the instant invention may employ receiver 64, transmitter 66, transceiver 62, and/or transponder units along with antennas and power supplies, in enclosures suitable for outdoor mounting in various locations in the vicinity of feedlot pens, or in other outdoor environments where animals to be monitored may be located. Such units will normally be mounted on poles or other elevated structures 62 as illustrated in FIG. 1 and may include, in alternate embodiments, separately or together, solar cells and other capabilities to enable operation from solar power without reliance on site-provided electrical power, and/or capabilities to operate from normal site-supplied electrical power; capabilities, as described further herein, to support precision location of animals within the feedlot or other animal care environment; and capabilities to monitor outdoor temperature and optionally other environmental parameters which may relate to the health and status of the animals being monitored, and data storage and processing capabilities. Some embodiments may also include still or video cameras and related transmission capabilities on elevated platforms to collect and transmit images continually, periodically, or upon command to aid in monitoring the health and status of animals.

Other embodiments of the instant invention may employ receiver/transmitter units in enclosures suitable for mounting in indoor environments within barns, kennels, stables, laboratories, and other animal production and animal care environments, and including, in alternate embodiments, separately or together, other features as described above for the outdoor mounted receiver/transmitter units.

Databases

Many embodiments of the instant invention employ data bases to maintain information on the identification codes for telesensors installed in or on specific animals and the corresponding human readable identification codes installed on the animals. In many cases, additional information such as receiving weight, breed, medication, origin, health related parameters, and other information useful for management of the animals may also be included in the same or associated databases. Such databases may be implemented in commercial packages such as Oracle or Microsoft's Access or SQL Server readily available from many sources. Such databases may be installed and maintained on one or more standard personal computers preferably networked via commercially available wired or wireless networking technologies with other computers, installation tools, receivers, transmitters, transceivers, modems, pagers, and other devices supporting implementation of alternate embodiments of the AAHMS system. Alteratively, such databases may also be implemented in whole or in part on portable or personal computer devices worn or carried, for example, by personnel responsible for, or supporting care of, the animals being monitored. In some embodiments, data pertinent to individual animals may be stored in memory capability integrated into the telesensor unit installed in the animal.

Central Processing and Control Unit(s)

Many embodiments of the instant invention employ a central processor and/or control units to support collection and management of information related to the health and status of the animals being monitored, and/or information related to the general management of the animals. In some embodiments, the central processing and control unit may also issue alerts automatically to pagers or other personnel alerting devices, personal digital assistants (PDAs), or personal computers with wireless communications capabilities carried by, worn by, or available to personnel responsible for animal care. In some embodiments, the central processor may employ voice synthesis software and other commercially available communications control hardware and software to provide a capability to generate synthesized voice transmissions to convey alert information to attendants using voice walkie-talkies and other personal radio devices as are already commonly available in feelot and other animal care operations. As noted above, such central processing and control units are preferably networked via commercially available wired or wireless networking technologies with other computers, installation tools, receivers, transmitters, transceivers, modems, pagers, and other devices supporting alternate implementations of the AAHMS system. In some embodiments, the central processing and control unit(s) may employ algorithms such as the “traveling salesman” algorithm in linear programming to develop and transmit efficient task schedules to animal attendants.

Conventional Computer Networking Capabilities

Many embodiments of the instant invention employ wired or wireless conventional computer networking hardware and software 72 to interconnect various components of the system. One skilled in the art of computer networking can select commercially available components as appropriate to support networking of the Central Processing and Control Unit(s), personnel alerting devices, PDAs, and other elements of the AAHMS as needed to tailor an embodiment for a particular application. In some embodiments, the networking may extend to the use of commercial telecommunications media (e.g., cell phones, local telephone company lines, long distance services) to enable transmission of health related parameters and alerts to an off-site location to enable off-site monitoring of the health and status of animals or use of a centralized monitoring capability for multiple sites. Commercial software packages (e.g., Windows NT and associated telecommunication packages) are available which support automated dialing of pager numbers or other phone numbers and transmission of data as scripted when certain conditions are met.

Personnel Alerting Devices

Many embodiments of the instant invention employ a personal alerting device 74 for animal caretaker personnel, comprising, in alternate embodiments, separately or together, a digital pager; a Personal Digital Assistant (PDA) or hand-held or palm computer or wrist-watch computer with wireless reception capabilities; and/or a walkie-talkie, cellular telephone, or other personal communications and signaling device. Although a simple alert with no further information would be useful, it is preferable in most applications, and supported by readily available commercial hardware and software, that the Personnel Alerting Devices supporting the instant invention include a capability to receive and display data (e.g., pen number, cage number) which identifies the sick animal and sufficient information on the animal's location to permit the attendant to proceed directly to the location of the animal to make additional diagnosis or render assistance or care as required. In some embodiments, the PDAs may include a transmit capability whereby the animal attendent can transmit data to the central processor and control unit to indicate the status of an animal, a task, or other information.

Portable Data Readout and Programming Units

Many embodiments of the instant invention employ hand-held wireless data readout units 76 having a capability to transmit a readout command to wireless telensors and/or tags as described above and elsewhere herein, and having a capability to receive data transmitted from said wireless telesensors and/or tags in response to said commands, and in alternate embodiments, having a capability to store, process, display, and re-transmit said information. In some embodiments, the data readout units will be combined with a capability to transmit commands and data to implement changes in the performance of individual telesensors so commanded. Examples of such changes include changing parameter thresholds at which an alarm is triggered, changing the time intervals at which parameter measurements are made or transmitted, or changing algorithms used to trigger alarm conditions.

Ambient Environment Sensor Units

In some embodiments, Ambient Environment Sensor Units (AESU) 80 may be employed to make measurements of parameters of the ambient environment, outdoor or indoor, within which animals being monitored by the AAHMS system are living. AESU elements used within some implementations include one or more sensors capable of performing measurements of one or more parameters of the ambient environment which may directly affect the health and status of the animals, or which may affect parameters measured by the telesensor units described above and later herein, which may affect the interpretation of data from the telesensor units, or which may affect the ability of telesensor units to accurately measure such parameters. Examples of ambient environment parameters which may be important in different implementations of the AAHMS for different applications include ambient temperature, humidity, wind velocity, solar radiance, atmospheric dust, and smoke. Thus, in various embodiments, the AESU may comprise one or more of the following commercially available sensors configured for remote readout: temperature sensors, humidity sensors (e.g., humidistats), anemometers (rotating cup, hot wire, etc.), solar irradiance sensors (e.g., solar cells, photometers), and dust and smoke sensors (e.g., light occlusion sensors, radiation attenuation sensors). Data from AESUs is typically transmitted to the Central Processing and Control Unit(s) via conventional computer networking devices.

Having thus described our invention and the manner of its use, it should be apparent to those skilled in the art that incidental changes may be made thereto that fairly fall within the scope of the following appended claims,

Wherein we claim:

1. A system for detecting at least one selected condition in an animal comprising:
at least one sensor associated with said animal, said sensor disposed to monitor at least one biometric parameter of said animal,
a transmitter coupled to said sensor, said transmitter providing a signal indicative of said biometric parameter,
a receiver of said signal, and,
indica for providing a reading of said biometric parameter.
